(19) United States
(12) Patent Application Publication
(10) Pub. No.: US 2010/0048713 A1
(43) Pub. Date: Feb. 25, 2010

(54) COMPOUNDS ACTING ON THE SEROTONIN TRANSPORTER

(75) Inventors: Ove Kjaer Wiborg, Arhus (DK); Henrik Amtoft Neubauer, Ega (DK)

Correspondence Address: DAVIS WRIGHT TREMAINE LLP - San Francisco, 505 MONTGOMERY STREET, SUITE 800, SAN FRANCISCO, CA, 94111, US

(73) Assignees: Aarhus Universitet, Arhus C (DK); Region Midtjylland, Viborg (DK)

(21) Appl. No.: 12/281,772

(22) PCT Filed: Jan. 8, 2007
(86) PCT No.: PCT/DK07/00009

§ 371 Date: Apr. 23, 2009

(30) Foreign Application Priority Data
Jan. 6, 2006 (DK) PA 2006 00030
Mar. 31, 2006 (DK) PA 2006 00461

(51) Int. Cl. A61K 31/16; A61K 31/10

(52) U.S. Cl. 514/609; 514/709

(57) ABSTRACT

The invention relates to new chemical compounds acting on the serotonin transporter (SERT), and associated pharmaceutical compositions, methods for use as therapeutic agents, and methods of preparation thereof. In particular the new chemical compounds are useful for the treatment of a variety of central nervous system (CNS) disorders—for example anxiety, depression, epilepsy, obsessive-compulsive disorders, migraine, cognitive disorders, sleep disorders, feeding disorders, panic attacks, disorders related to withdrawl from drug abuse, schizophrenia, or the like, or in the treatment of gastrointestinal disorders such as irritable bowel syndrome.

All patent and non-patent references cited in the present application are also hereby incorporated by reference in their entirety.

### FIELD OF INVENTION

The present invention relates to new chemical compounds acting on the serotonin transporter (SERT), and associated pharmaceutical compositions, methods for use as therapeutic agents, and methods of preparation thereof.

### BACKGROUND OF INVENTION

The Serotonin Transporter SERT

Serotonin is localized in the central and peripheral nervous systems and is known to affect many types of conditions including psychiatric disorders, motor activity, feeding behavior, sexual activity, and neuroendocrine regulation among others.

Serotonergic neurotransmission is modulated by clearance of serotonin (5-hydroxytryptamine or 5-HT). The clearance of 5-HT from the synaptic cleft is maintained by the serotonin transporter (SERT). The transporter therefore affects the magnitude and duration of the signalling, and thus plays a key role in the spatio temporal fine tuning of serotonergic neurotransmission

The serotonin transporter (SERT), which belongs to a family of sodium/chloride-dependent transporters, is the major pharmacological target in the treatment of several clinical disorders, including depression and anxiety. Activation of a low affinity allosteric site on SERT modulates the ligand affinity at the high affinity binding site. Serotonin (5-HT), as well as some SERT inhibitors possesses affinity for both sites.

SERT is a well established molecular target of drugs of abuse (cocaine and amphetamines), as well as a number of high-affinity antidepressants. Multiple classes of antidepressants including tricyclic antidepressants, 5-HT selective reuptake inhibitors and antidepressants with dual actions are directed towards SERT. They enhance serotonergic neurotransmission by inhibiting 5-HT reuptake in a competitive manner with inhibitory constants in the low nanomolar range (Barker and Blakely, 1995; Owens et al., 1997; Tatsumi et al., 1997).

Dissociation of the tricyclic imipramine from platelet membranes is attenuated in the presence of 5-HT (Wennogle and Meyerson, 1982; Wennogle and Meyerson, 1985) suggesting that 5-HT acts at a site distinct from the imipramine binding site. Several high affinity SERT inhibitors (citalopram, paroxetine, sertraline, imipramine) can also act as allosteric ligands (Plenge and Mellerup, 1985; Plenge et al., 1991). The affinity-modulating or allosteric site has been shown to be present at all three monoamine transporters, which in addition to SERT also includes transporters for dopamine and norepinephrine (Plenge and Mellerup, 1997).

The interaction with the allosteric binding site is specific for SERT as supported by several findings. Strong effects on dissociation rates are only exerted by a subset of drugs tested (Plenge et al., 1991; Chen et al., 2005b). The effect is stereo selective, as some enantiomers have different potencies (Plenge et al., 1991). Species differences have been reported, concerning the allosteric potency of specific drugs (Plenge et al., 1991). A species scanning mutagenesis study comparing human and chicken SERT revealed that nine residues in the C-terminal part were an important part of an allosteric mechanism that mediated the allosteric effect of e.g. escitalopram (Neubauer et al, 2006).

Depression

Depression is a common, life-disrupting, potentially lethal illness that can affect both sexes and all ages. Untreated major depression remains a serious public health problem and its incidences are staggering. Its peak onset is in the early adult years. Suicide occurs in as many as 15% of patients with depression, especially those with recurrent episodes and hospitalizations. Therefore it becomes evident that treatment of depression is a matter of prime importance. Depression has no single cause; often, it results from a combination of factors. Whatever its cause, depression is not just a state of mind; it is related to physical changes in the brain, and connected to an imbalance of neurotransmitters. Among the most important neurotransmitters related with depression are serotonin (5-HT), norepinephrine (NE), and dopamine (DA). Serotonin plays a very important role in the mood disorders, especially in anxiety and depression, aggression and impulsivity. Regulation of the mood disorders is possible either by agonistic or antagonistic action on a certain type of the serotonin receptors.

Serotonin-selective reuptake inhibitors (SSRIs), such as fluoxetine (PROZAC (E), have traditionally been the mainstay of treatment for clinical depression-replacing the more toxic tricyclic antidepressants (TCAs). SSRIs have a more favourable adverse reaction profile in comparison to the TCAs and are much easier to tolerate. SSRIs exert their therapeutic effect by blocking the reuptake of serotonin into the presynaptic nerve terminal, thus increasing the synaptic concentration of serotonin. It is also believed that SSRIs increase the efficacy of the serotonin (5-HT) neurons by desensitizing 5-HT autoreceptors located on the presynaptic 5-HT nerve terminals. The ability of the 5-HT autoreceptors to inhibit the release of 5-HT decreases after long-term treatment with SSRIs, with the net effect being that a greater amount of 5-HT is released per impulse.

Unfortunately, there is a delay in the effect of SSRIs ranging from three to four weeks, or even longer, from the onset of treatment. Symptoms may sometimes even worsen during the first weeks of treatment. In order to treat the patient during the delayed effect of SSRI, additional antidepressants are used to augment the SSRI therapy by co-administration of compounds stabilizing the mood of the patient—such as for example lithium carbonate ortriiodothyronin or by the use of electroshock.

There is thus a need for compounds which can be administered in combination with anti-depressant drugs in order to decrease the delay in the effect of a therapeutic anti-depressant drug.

Drug Abuse

Cocaine binds to the SERT, dopamine transporter (DAT) and norepinephrin transporter (NET) and directly prevents the re-uptake of dopamine, serotonin, and norepinephrine into pre-synaptic neurons (Heikkila et al., 1975, Biochem Pharmacol 24(8):847-852; Reith et al., 1986, Biochem Pharmacol 35(7):1123-1129; Ritz et al., 1987, Science 237:1219-1223). Inhibition of re-uptake subsequently elevates the synaptic concentrations of each of these neurotransmitters.

Cocaine abuse is one of the greatest concerns of the world today, and has therefore become a focus of medical, social and political leaders. Cocaine is one of the most addictive substances known, and addicts may lose their ability to function at work or in interpersonal situations.

There is thus a need for compounds that act as antagonists at these transporters inhibiting the binding of cocain to the transporters without affecting the re-uptake of serotonin, dopamine, and norepinephrin, respectively.

### SUMMARY OF INVENTION

In one aspect is disclosed a compound for use as a medicament;
wherein said compound has the general formula A

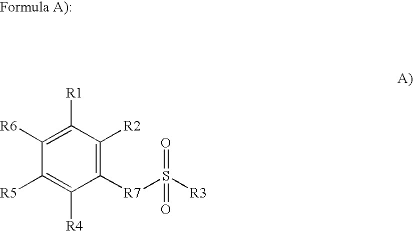

wherein R1, R2, R3, R4, R5, R6 are independently selected from any of the following chemical groups:
H, F, Cl, Br, I, hydroxy, alkoxy, alkenoxy, aryloxy, carbamoyl, carboxyl, thiol groups, alkyl sulfide, aryl sulfide, sulfone, sulfonyl, sulfoxide, amine, alkylamine, dialkylamine, arylamine, alkylarylamine, diarylamine, N-oxides (for example nitro-), mercapto, cyano,
a hydrocarbon substituent selected from the group consisting of:
alkyl, substituted alkyl,
cyclic alkyl, substituted cyclic alkyl,
aryl, substituted aryl,
alkenyl, substituted alkenyl,
alkynyl, substituted alkynyl,
aralkyl, substituted aralkyl,
heterocyclyl, substituted heterocyclyl,
heterocyclylalkyl, substituted heterocyclylalkyl,
alkylaminoalkyl, substituted alkylaminoalkyl,
dialkylaminoalkyl, substituted dialkylaminoalkyl,
heterocyclyloxyalkyl, substituted heterocyclyloxyalkyl,
arylaminoalkyl, substituted arylaminoalkyl,
heterocyclylaminoalkyl, substituted heterocyclylaminoalkyl,
alkylaminoalkoxy, substituted alkylaminoalkoxy,
dialkylaminoalkoxy, substituted dialkylaminoalkoxy,
heterocyclyloxy, and substituted heterocyclyloxy;
and wherein —R7- represents NH or O or CH₂ or is a single bond;
and wherein either one or none of the following pairs of R groups form part of an additional benzene ring:
(R1 and R2)
(R1 and R6)
(R5 and R6)
(R4 and R5);
wherein said additional benzene ring is optionally a substituted benzene ring;

with the provisos for formula A that:

if R7 is NH, then R3 does not comprise a five-sided organic ring; and

R6 does not comprise more than three organic ring structures; and

if R3 comprises a five-sided organic ring, then R3 does not comprise a six-sided organic ring; and

if R6 comprises a five-sided organic ring, then R6 does not comprise a six-sided organic ring; and

if R6 comprises a linear carbon chain or substituted linear carbon chain, then said carbon chain is C₁₋₇.

Or wherein the compound has the general formula B

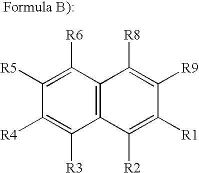

wherein R1, R2, R3, R4, R5, R6 are as defined for the general formula A, and

wherein R8 and R9 are independently selected from any of the possibilities for R1 described herein, preferably NH₂, NO₂, CH₃, CN, Cl, COOH, OH, NO₂, or OH, such as wherein the compound is selected from the group consisting of: naphthalene-1,5-diamine, 1-fluoro-4-nitro-naphthalene, 8-Nitro-naphthalene-1-carboxylic acid, 1-hydroxy-naphthalene-2-carboxylic acid, 2-Methyl-1-nitro-naphthalene, 2-hydroxy-naphthalene-1-carboxylic acid, 1-nitro-naphthalene, napthalen-1-ylamine, naphthalene-1-carbonitrile and 1-chloro-naphthalene,

or wherein the compound has the general formula C

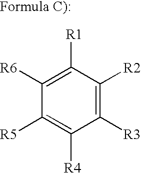

wherein R1, R2, R3, R4, R5, R6 are as defined for the general formula A, wherein R2-R6 of said compound falling under formula C are preferably H and/or wherein said compound falling under formula C is preferably selected from the group consisting of: benzeneboronic acid, phenyl-phosphonic acid, benzoic acid, benzamide and acetophenone.

Any of these compounds can be used as a medicament, such as for treatment of a variety of central nervous system (CNS) disorders—for example anxiety, depression, epilepsy, obsessive-compulsive disorders, migraine, cognitive disorders, sleep disorders, feeding disorders, panic attacks, disorders relating to withdrawal from drug abuse, schizophrenia, or the like, or in the treatment of gastrointestinal disorders such as irritable bowel syndrome.

Therefore, this invention provides compounds which are useful as therapeutic agents, for example in the treatment of a variety of central nervous system disorders related to or affected by SERT.

In another aspect of the present invention, the compounds according to the present invention can be used in therapeutic methods and pharmaceutical compositions, which can for example be useful for the treatment of central nervous system disorders related to or affected by SERT.

In another aspect, it has been found by the inventors of the present invention that the compounds falling under Formula I may be administered in combination with other anti-depressant compounds, such as selective serotonin reuptake inhibitors (SSRIs), in order to speed up the onset of the anti-depressant effect in a synergistic manner.

### DETAILED DESCRIPTION OF THE INVENTION

Compounds

One aspect of the present invention relates to a compound according to formula A for use as a medicament;

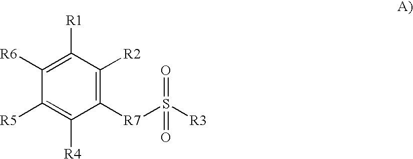

wherein —R7- represents NH or O or CH₂ or is a single bond;
and furthermore wherein R1, R2, R3, R4, R5, R6 are independently selected from any of the following chemical groups:
H, F, Cl, Br, I, hydroxy, alkoxy, aryloxy, carbamoyl, carboxyl, thiol groups, alkyl sulfide, aryl sulfide, sulfone, sulfonyl, sulfoxide, amine, alkylamine, dialkylamine, arylamine, alkylarylamine, diarylamine, N-oxides (for example nitro-), mercapto, cyano,
a hydrocarbon substituent selected from the group consisting of:
alkyl, substituted alkyl,
cyclic alkyl, substituted cyclic alkyl,
aryl, substituted aryl,
alkenyl, substituted alkenyl,
alkynyl, substituted alkynyl,
aralkyl, substituted aralkyl,
heterocyclyl, substituted heterocyclyl,
heterocyclylalkyl, substituted heterocyclylalkyl,
alkylaminoalkyl, substituted alkylaminoalkyl,
dialkylaminoalkyl, substituted dialkylaminoalkyl,
heterocyclyloxyalkyl, substituted heterocyclyloxyalkyl,
arylaminoalkyl, substituted arylaminoalkyl,
heterocyclylaminoalkyl, substituted heterocyclylaminoalkyl,
alkylaminoalkoxy, substituted alkylaminoalkoxy,
dialkylaminoalkoxy, substituted dialkylaminoalkoxy,
heterocyclyloxy, and substituted heterocyclyloxy,

and/or furthermore wherein either one or none of the following pairs of R groups form part of an additional benzene ring:
(R1 and R2)
(R1 and R6)
(R5 and R6)
(R4 and R5);

by which is preferably meant that formula A encompasses the following chemical structures:

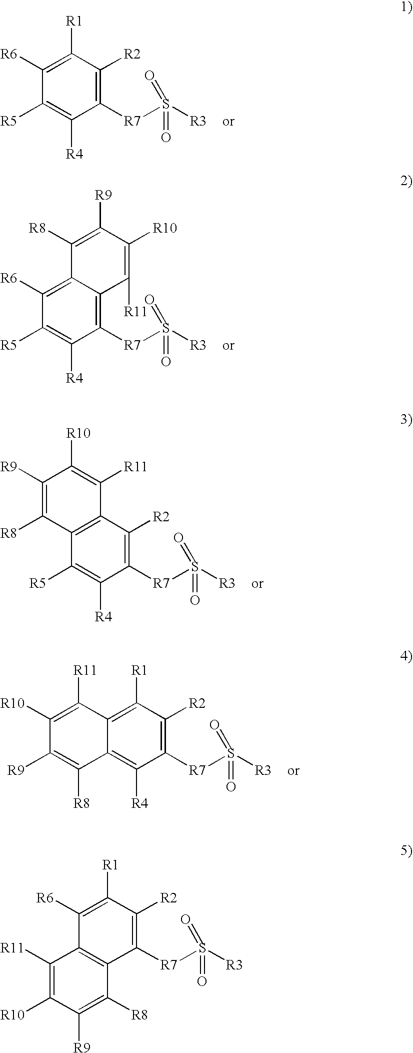

wherein R8-R11 are independently selected from the same chemical groups specified herein as suitable for R1.

In one embodiment of the present invention, Formula A encompasses the above embodiments 1), 2) and 5).

In one embodiment of the present invention, it is preferred that one or more of the following provisos are fulfilled:

1) if R7 is NH, then R3 does not comprise a five-sided organic ring; and/or

2) R6 is not an ester group and/or

3) R6 does not comprise more than three organic ring structures; and/or

4) if R3 comprises a five-sided organic ring, then R3 does not comprise a six-sided organic ring; and/or

5) if R6 comprises a five-sided organic ring, then R6 does not comprise a six-sided organic ring; and/or

6) if R6 comprises a linear carbon chain or substituted linear carbon chain, then said carbon chain is C₁₋₇.

Preferably, 2-6 of the above provisos are fulfilled, such as 3-6, for example 4-6, such as 5-6 of the above provisos are fulfilled, for example in the following combinations:

(1+3+4+6)

(3+4+5+6)

(2+4+5+6)

In a further embodiment of the present invention, the compounds according to the present invention have a structure according to formulae B or C, as follows:

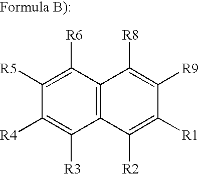

wherein R1-R6 are independently selected from any of the possibilities for R1-R6 as disclosed herein, and/or

wherein R8 and R9 are independently selected from any of the possibilities for R1 described herein, preferably NH₂, NO₂, CH₃, CN, Cl, COOH, OH, NO₂, or OH, such as wherein the compound is selected from the group consisting of: naphthalene-1,5-diamine, 1-fluoro-4-nitro-naphthalene, 8-Nitro-naphthalene-1-carboxylic acid, 1-Hydroxy-naphthalene-2-carboxylic acid, 2-Methyl-1-nitro-naphthalene, 2-Hydroxy-naphthalene-1-carboxylic acid, 1-nitro-naphthalene, napthalen-1-ylamine, naphthalene-1-carbonitrile and 1-chloro-naphthalene.

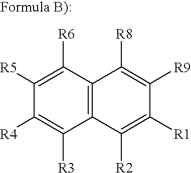

wherein R1-R6 are independently selected from any of the possibilities for R1-R6 as disclosed herein, and/or

wherein R2-R6 of said compound falling under formula C are preferably H and/or wherein said compound falling under formula C is preferably selected from the group consisting of: benzeneboronic acid, phenyl-phosphonic acid, benzoic acid, benzamide and acetophenone.

In one embodiment, the compound according to the present invention has a molecular weight below 10,000 Da, such as below 9,000 Da, for example below 8 kDa, such as below 7 kDa, for example below 7 kDa, such as below 6 kDa, for example below 5 kDa, such as below 4 kDa, for example below 3 kDa, such as below 2 kDa, for example below 1500 Da, such as below 1200 Da, for example below 1000 Da, such as below 900 Da, for example below 800 Da, such as below 700 Da, for example below 600 Da, such as below 500 Da, such as below 400 Da, for example below 300 Da, such as below 200 Da, for example below 150 Da, such as below 100 Da.

### Embodiments of R7

In one embodiment of the present invention, —R7- is a single bond.

In another embodiment of the present invention, —R7- is O.

In another embodiment of the present invention, —R7- is NH.

In another embodiment of the present invention, —R7- is CH₂.

### Embodiments of R1

In the compounds according to the present invention, R1 can be selected from the group consisting of:
H, F, Cl, Br, I, hydroxy, alkoxy, alkenoxy, aryloxy, carbamoyl, carboxyl, thiol, alkyl sulfide, aryl sulfide, sulfone, sulfonyl, sulfoxide, amine, alkylamine, dialkylamine, arylamine, alkylarylamine, diarylamine, N-oxide, mercapto, cyano, alkyl, substituted alkyl,
cyclic alkyl, substituted cyclic alkyl,
aryl, substituted aryl,
alkenyl, substituted alkenyl,
alkynyl, substituted alkynyl,
aralkyl, substituted aralkyl,
heterocyclyl, substituted heterocyclyl,
heterocyclylalkyl, substituted heterocyclylalkyl,
alkylaminoalkyl, substituted alkylaminoalkyl,
dialkylaminoalkyl, substituted dialkylaminoalkyl,
heterocyclyloxyalkyl, substituted heterocyclyloxyalkyl,
arylaminoalkyl, substituted arylaminoalkyl,
heterocyclylaminoalkyl, substituted heterocyclylaminoalkyl,
alkylaminoalkoxy, substituted alkylaminoalkoxy,
dialkylaminoalkoxy, substituted dialkylaminoalkoxy,
heterocyclyloxy, and substituted heterocyclyloxy,

In one embodiment of the present invention, if R1 comprises a linear carbon skeleton, then said linear carbon skeleton is C₁₋₂₅, such as C₂₋₁₅ or C₁₋₂₀, for example C₁₋₁₅, such as C₁₋₁₀, for example C₁₋₈, such as C₁₋₅ or C₁₋₃

In another embodiment of the present invention, R1 is selected from the group consisting of: H, F, Cl, Br, I, hydroxyl and alkoxy.

In another embodiment of the present invention, R1 is selected from the group consisting of: aryloxy, carbamoyl, carboxyl, thiol, alkyl sulphide, aryl sulphide, sulfone, sulfonyl, sulfoxide or amine.

In another embodiment of the present invention, R1 is selected from the group consisting of: alkylamine, dialkylamine, arylamine, alkylarylamine, diarylamine, or an N-oxide.

In another embodiment of the present invention, R1 is selected from the group consisting of: alkyl, substituted alkyl, cyclic alkyl, and substituted cyclic alkyl.

Thus, in one embodiment, R1 is an optionally substituted alkyl. Said optionally substituted alkyl can be a straight chain alkyl selected from the group consisting of: methyl, ethyl, propyl, butyl, pentyl, hexyl, heptyl, octyl, nonyl, decyl, undecyl, and dodecyl. Another embodiment of said optionally substituted alkyl is a branched chain alkyl, such as selected from the group consisting of:

—CH(CH₃)₂,

—CH(CH₃)(CH₂CH₃),

—CH(CH₂CH₃)₂,

—C(CH₃)₃,

—C(CH₂CH₃)₃,

—CH₂CH(CH₃)₂,

—CH₂CH(CH₃)(CH₂CH₃),

—CH₂CH(CH₂CH₃)₂,

—CH₂C(CH₃)₃,

—CH₂C(CH₂CH₃)₃,

—CH(CH₃)CH(CH₃)(CH₂CH₃),

—CH₂CH₂CH(CH₃)₂,

—CH₂CH₂CH(CH₃)(CH₂CH₃),

—CH₂CH₂CH(CH₂CH₃)₂,

—CH₂CH₂C(CH₃)₃,

—CH₂CH₂C(CH₂CH₃)₃,

—CH(CH₃)CH₂CH(CH₃)₂,

—CH(CH₃)CH(CH₃)CH(CH₃)CH(CH₃)₂, and

—CH(CH₂CH₃)CH(CH₃)CH(CH₃)(CH₂CH₃).

In another embodiment of the present invention, R1 is selected from the group consisting of: aryl or substituted aryl.

In another embodiment of the present invention, R1 is selected from the group consisting of: alkenyl, substituted alkenyl, alkynyl, substituted alkynyl, aralkyl, or substituted aralkyl.

In another embodiment of the present invention, R1 is selected from the group consisting of: heterocyclyl, substituted heterocyclyl, heterocyclylalkyl, substituted heterocyclylalkyl, alkylaminoalkyl, substituted alkylaminoalkyl, dialkylaminoalkyl, substituted dialkylaminoalkyl, heterocyclyloxyalkyl, or substituted heterocyclyloxyalkyl.

In another embodiment of the present invention, R1 is selected from the group consisting of: arylaminoalkyl, substituted arylaminoalkyl, heterocyclylaminoalkyl, or substituted heterocyclylaminoalkyl.

In another embodiment of the present invention, R1 is selected from the group consisting of: alkylaminoalkoxy, substituted alkylaminoalkoxy, dialkylaminoalkoxy, substituted dialkylaminoalkoxy, heterocyclyloxy, or substituted heterocyclyloxy.

In another embodiment of the present invention, R1 is selected from the group consisting of: OH, NH₂, halogen, methyl, H.

In another embodiment of the present invention, R1 is selected from the group consisting of: H, NH2, OH, a halogen, Cl, CH3, C2H5, a phenyl group, Cl,

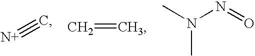

(in any orientation),

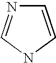

(in any

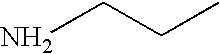

orientation),

In another embodiment of the present invention, R1 is H.

Thus, R1 can be an optionally substituted cycloalkyl. Said optionally substituted cycloalkyl can for example be selected from the group consisting of: cyclopropyl, cyclobutyl, cyclopentyl, cyclohexyl, cycloheptyl, and cyclooctyl and rings substituted with straight and branched chain alkyl groups, such as any of straight and branched chain alkyl groups disclosed herein. The optionally substituted cycloalkyl can also be a C₁-C₂₆ cycloalkyl group, such as a C₁-C₁₅ cycloalkyl group, for example a C₁-C₁₂ cycloalkyl group, such as a C₁-C₉ cycloalkyl group, for example a C₅-C₉ cycloalkyl group.

Thus, R1 can be an optionally substituted aralkyl group, such as selected from the group consisting of: benzyl, diphenylmethyl, 1-phenylethyl(-CH(C₆H₅)(CH₃)), 2-phenylethyl group, and 2-naphthylethyl group.

Thus, R1 can be an optionally substituted alkyl residue consisting of a C₁-C₄₀ alkyl group, such as a C₁-C₃₀ alkyl group, for example a C₁-C₂₀ alkyl group, such as a C₁-C₁₅ alkyl group or a C₅-C₁₅ alkyl group, such as a C₁-C₁₀ alkyl group or a C₅-C₁₀ alkyl group, such as a C₁-C₈ alkyl group or a C₅-C₈ alkyl group, such as a C₁-C₅ alkyl group or a C₄-C₉ alkyl group.

Any of the alkyl groups disclosed herein may be substituted with an aryl or heteroaryl residue.

For example, the alkyl substituted with an aryl or heteroaryl residue can be selected from the group consisting of linear (C1-C10) alkyls, branched (C4-C10) alkyls, cyclic (C5-C10) alkyls, such as a methyl group, ethyl group, propyl group, such as a n-propyl group and an isopropyl group, butyl group, such as n-butyl group, isobutyl group, t-butyl group, n-amyl group, pentyl group, such as neopentyl group, cyclopentyl group, hexyl group, such as n-hexyl group, cyclohexyl group, heptyl group, octyl group, such as n-octyl group, nonyl group, such as n-nonyl group, decyl group, such as n-decyl group, undecyl group, dodecyl group, and mentyl group.

Furthermore, any of the alkyl groups disclosed herein can be an optionally substituted C₅-C₂₀ alkyl group which, when substituted, is substituted with one or more of a halogen atom, such as fluorine atom, a chlorine atom, a bromine atom, and an iodine atom, an alkoxy group, such as a (C1-C4) alkoxy group such as methoxy group, ethoxy group, n-propoxy group, t-butoxy group, an aryloxy group, such as phenoxy, an alkylthio group, such as n-propylthio and t-butylthio, and an arylthio group, such as phenylthio.

In another embodiment, R1 is an optionally substituted alkenyl. Said optionally substituted alkenyl can be a straight or branched chain or a cyclic group, such as vinyl,
—CH═C(H)(CH₃), —CH═C(CH₃)₂, —C(CH₃)═C(H)₂, —C(CH₃)═C(H)(CH₃), —C(CH₂CH₃)═CH₂, cyclohexenyl, cyclopentenyl, cyclohexadienyl, butadienyl, pentadienyl, and hexadienyl.

The term “substituted alkenyl group” can in one embodiment include alkenyl groups in which a non-carbon or non-hydrogen atom is bonded to a carbon double bonded to another carbon and those in which one of the non-carbon or non-hydrogen atoms is bonded to a carbon not involved in a double bond to another carbon.

Any of the alkynyl groups disclosed herein can for example be a straight or branched chain group such as —CC(H), —CC(CH₃), —CC(CH₂CH₃), —C(H₂)CC(H), —C(H)₂CC(CH₃), and —C(H)₂CC(CH₂CH₃).

Examples of suitable substituted alkynyl groups include alkynyl groups in which a non-carbon or non-hydrogen atom is bonded to a carbon triple bonded to another carbon and those in which a non-carbon or non-hydrogen atom is bonded to a carbon not involved in a triple bond to another carbon.

It should also be understood by one skilled in the art that any of the R1 groups disclosed herein may be a substituted hydrocarbon substituent, such as any of the groups disclosed herein substituted with one or more, such as two, three, four, five, six, seven, eight, nine and ten, of a halogen atom such as F, Cl, Br, and I; and oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as in trialkylsilyl groups, dialkylarylsilyl groups, alkyldiarylsilyl groups, and triarylsilyl groups. Thus, the substituted hydrocarbon substituent can be substituted with one or more, such as two, three, four, five, six, seven, eight, nine and ten, of a heteroatom, such as oxygen in carbonyl, carboxyl, and ester groups; nitrogen in groups such as imines, oximes, hydrazones, and nitriles. Or, for example, the substituted hydrocarbon substituent can contain a hydroxyl, alkoxy, aryloxy group, or heterocyclyloxy group. Or, for example, the substituted hydrocarbon substituent can contain an amine, alkylamine, dialkylamine, arylamine, (alkyl)(aryl)amine, diarylamine, heterocyclylamine, (alkyl)(heterocyclyl)amine, (aryl)(heterocyclyl)amine, or diheterocyclylamine group. Or, for example, the substituted hydrocarbon substituent is an aliphatic functional group substituted with an aryl group such as any of the (C6-C12) aryl groups disclosed herein. Or, for example, the substituted hydrocarbon substituent can be a substituted aryl group (for example an aralkyl group), which can be substituted or non-substituted.

### Embodiments of R2

R2 is independently selected from any of the embodiments described above in the section entitled: “Embodiments of R1”

### Embodiments of R3

R3 is independently selected from any of the embodiments described above in the section entitled: “Embodiments of R1”

In a preferred embodiment R3 is an alkoxy or alkenoxy, such as an methoxy, ethoxy, n-propoxy, iso-propoxy, n-butoxy, tert-butoxy, sec-butoxy, n-pentoxy, n-hexoxy, 1,2-dimethylbutoxy, and the like. In particular R3 is alkoxy or alkenoxy as defined above in formula A.

### Embodiments of R4

R4 is independently selected from any of the embodiments described above in the section entitled: “Embodiments of R1”

### Embodiments of R5

R5 is independently selected from any of the embodiments described above in the section entitled: “Embodiments of R1”

### Embodiments of R6

R6 is independently selected from any of the embodiments described above in the section entitled: “Embodiments of R1”

### Other Examples of Embodiments of the Present Invention

In one embodiment of the present invention, at least 2, such as at least 3, for example at least 4, such as at least 5 of R1-R6 are selected from the group consisting of: H, OH, NH, and a halogen.

In another embodiment of the present invention, at least 2, such as at least 3, for example at least 4, such as at least 5 of R1-R6 are H.

In another embodiment of the present invention, at least 2, such as at least 3, for example at least 4, such as at least 5 of R1-R6 are selected from the group consisting of: H, OH, NH, and a halogen.

In another embodiment of the present invention, at least 2, such as at least 3, for example at least 4, such as at least 5 of R1-R6 have a molecular weight of less than 50, such as less than 40, for example less than 30, such as less than 20, for example less than 15, such as less than 10, for example less than 8 (preferably less than 20).

In one embodiment of the present invention:

a) R1 is selected from the group consisting of: H, NH₂, OH, a halogen, Cl, CH₃, C₂H₅

and b) R2 is selected from the group consisting of: H, NH₂, OH, a halogen, Cl, CH₃, C₂H₅

and c) R3 is selected from the group consisting of: a halogen, a phenyl group, Cl, H, NH₂, OH, CH₃, C₂H₅

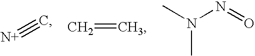

(in any orientation),

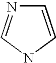

(in any orientation),

and d) R4 is selected from the group consisting of: H, NH₂, OH, a halogen, Cl, CH₃, C₂H₅

and e) R5 is selected from the group consisting of: H, NH₂, OH, a halogen, Cl, CH₃, C₂H₅

and f) R6 is selected from the group consisting of: a C1-4 alkyl group, H, NH2, H, NH₂, OH, a halogen, Cl,

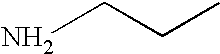

In yet another embodiment the compound of formula A has a general formula A′

In another embodiment of the present invention, the compounds of the present invention are selected from the group consisting of: benzeneboronic acid, benzoic acid, phenyl-phosphonic acid, acetophenone, diphenyl sulfone, benzamide, 5-dimethylaminonaphtalene-1-sulfonyl chloride, 1-amino-8-naphtalenesulfonic acid, 4amino-1-naphtalenesulfonic acid, naphthalene-1,5-diamine, 4-amino-3-hydroxy-1-naphthalenesulfonic acid, naphthalene-1-carbonitrile, naphthalene-1-ylamine, 2-methyl-1-nitro-naphtalene, 8-nitro-naphthalene-1-carboxylic acid, 2-hydroxy-naphthalene-1-carboxylic acid, 1-fluoro-4-nitro-naphthalene, 1-hydroxy-naphthalene-2-carboxylic acid,

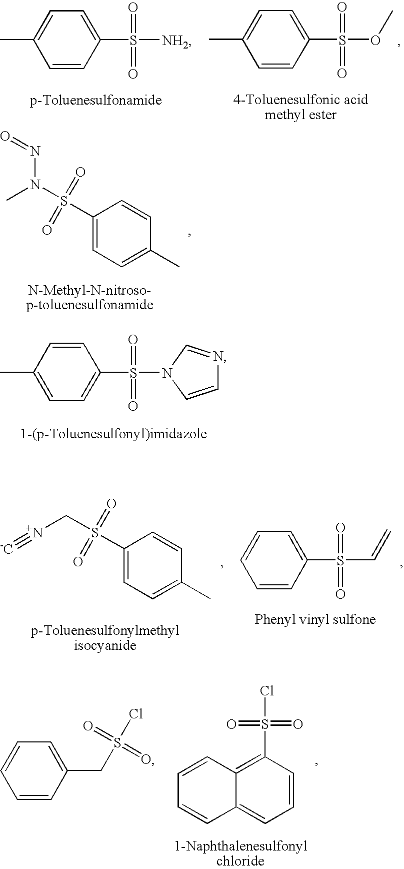

1-naphthalenesulfonic acid,

Naphthalene-1-sulfonic acid isopropyl ester,

Naphthalene-1-sulfonic acid isopropenyl ester,

Naphthalene-1-sulfonic acid ethyl ester,

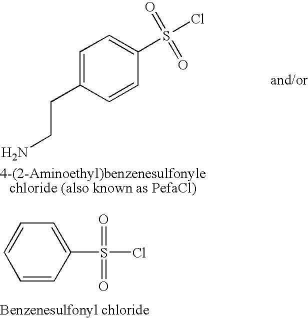

In another embodiment of the present invention the compounds of the present invention are selected from the group consisting of:

diphenyl sulfone, 5-dimethylaminonaphtalene-1-sulfonyl chloride, 1-amino-8-naphtalenesulfonic acid, 4-amino-1-naphtalenesulfonic acid, 4-amino-3-hydroxy-1-naphthalenesulfonic acid,

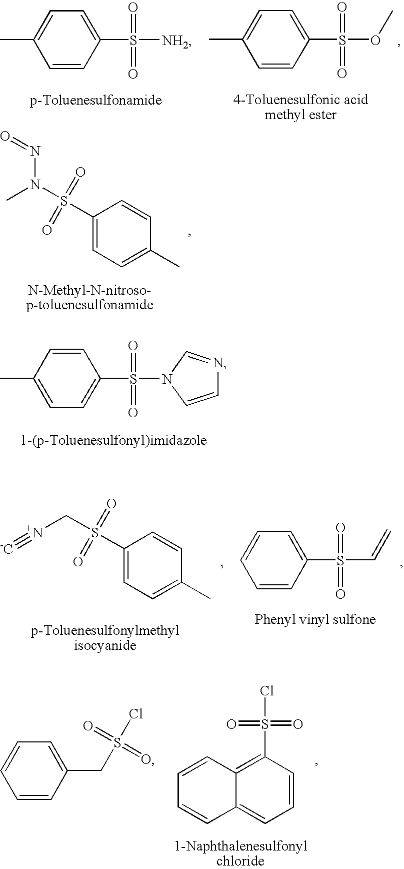

1-naphthalenesulfonic acid,

Naphthalene-1-sulfonic acid isopropyl ester,

Naphthalene-1-sulfonic acid isopropenyl ester,

Naphthalene-1-sulfonic acid ethyl ester,

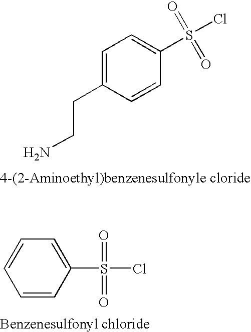

In yet another embodiment of the present invention the compounds of the present invention are selected from the group consisting of:

naphthalene-1,5-diamine, naphthalene-1-carbonitrile, naphthalene-1-ylamine, 2-methyl-1-nitro-naphtalene, 8-nitro-naphthalene-1-carboxylic acid, 2-hydroxy-naphthalene-1-carboxylic acid, 1-fluoro-4nitro-naphthalene, 1-hydroxy-naphthalene-2-carboxylic acid, 4-amino-3-hydroxy-1-naphtalenesulfonic acid.

In another embodiment of the present invention the compounds of the present invention are selected from the group consisting of:

Benzeneboronic acid, acetophenone, 4-amino-1-naphthalenesulfonic acid, 1-(p-toluensulfonyl)imidazole, 1-hydroxy-naphtaIene-2-carboxylic acid. 2-hydroxy-naphthalene-1-carboxylic acid, 4-amino-3-hydroxy-1-naphthalenesulfonic acid.

In a further embodiment it is appreciated that any of the listed compounds may be used separately, such as benzeneboronic acid, for example benzoic acid, such as phenyl-phosphonic acid, for example acetophenone, such as diphenyl sulfone, for example benzamide, such as 5-dimethylaminonaphtalene-1-sulfonyl chloride, for example 1-amino-8-naphtalenesulfonic acid, such as 4-amino-1-naphtalenesulfonic acid, for example naphthalene-1,5-diamine, such as 4amino-3-hydroxy-1-naphthalenesulfonic acid, for example naphthalene-1-carbonitrile, such as naphthalene-1-ylamine, for example 2-methyl-1-nitro-naphtalene, such as 8-nitro-naphthalene-1-carboxylic acid, for example 2-hydroxy-naphthalene-1-carboxylic acid, such as 1-fluoro-4-nitro-naphthalene, for example 1-hydroxy-naphthalene-2-carboxylic acid, such as

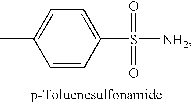

for example

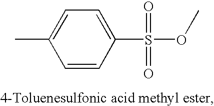

such as

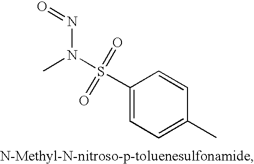

for example N-methyl-N-nitroso-p-toluensulfonamide, such as 1-(p-Toluensulfonyl)imidazole, for example p-Toluensulfonylmethyl isocyanide, such as phenyl vinyl sulfone, for example 1-naphthalenesulfonyl chloride, such as 1-naphthalenesulfonylic acid, Naphthalene-1-sulfonic acid isopropyl ester, Naphthalene-1-sulfonic acid isopropenyl ester, or Naphthalene-1-sulfonic acid ethyl ester, for example 4-(2-Aminoethyl)benzenesulfonyle cloride such as benzenesulfonyl chloride.

Definitions of Embodiments Included within the Meaning of the Above-Mentioned Chemical Groupings

“Alkyl” as used herein includes straight chain alkyl groups such as methyl, ethyl, propyl, butyl, pentyl, hexyl, heptyl, octyl, nonyl, decyl, undecyl, dodecyl and the like. “Alkyl” also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)CH(CH₃)₂, —CH(CH₂CH₃)CH(CH₃)CH(CH₃)(CH₂CH₃), and others.

The aliphatic residue can be an optionally substituted linear aliphatic residue or an optionally substituted branched aliphatic residue. The aliphatic residue can also be an optionally substituted cyclic alkyl. “Cyclic alkyl” includes groups such as cyclopropyl, cyclobutyl, cyclopentyl, cyclohexyl, cycloheptyl, and cyclooctyl and such rings substituted with straight and branched chain alkyl groups as defined above. also includes polycyclic alkyl groups such as, but not limited to, adamantyl norbornyl, and bicyclo[2.2.2]octyl and such rings substituted with straight and branched chain alkyl groups as defined above. Thus, unsubstituted alkyl groups includes primary alkyl groups, secondary alkyl groups, and tertiary alkyl groups. Unsubstituted alkyl groups may be bonded to one or more carbon atom(s), oxygen atom(s), nitrogen atom(s), and/or sulfur atom(s) in a ligand.

The cyclic aliphatic residue can e.g. comprise or consist of a C₅-C₁₆ cycloalkyl group. Shorter chain lengths can also occur, typically when the cycloalkyl is substituted with an aryl or heteroaryl residue.

“Substituted alkyl” refers to an unsubstituted alkyl group as defined above in which one or more bonds to a carbon(s) or hydrogen(s) are replaced by a bond to non-hydrogen and non-carbon atoms such as, but not limited to, a halogen atom in halides such as F, Cl, Br, and I; and oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as in trialkylsilyl groups, dialkylarylsilyl groups, alkyldiarylsilyl groups, and triarylsilyl groups; and other heteroatoms in various other groups.

Substituted alkyl groups also include groups in which one or more bonds to a carbon(s) or hydrogen(s) atom is replaced by a bond to a heteroatom such as oxygen in carbonyl, carboxyl, and ester groups; nitrogen in groups such as imines, oximes, hydrazones, and nitrites. Substituted alkyl groups also include, among others, alkyl groups in which one or more bonds to a carbon or hydrogen atom is/are replaced by one or more bonds to a halogen atom. Other substituted alkyl groups include those in which one or more bonds to a carbon or hydrogen atom is replaced by a bond to an oxygen atom such that the substituted alkyl group contains a hydroxyl, alkoxy, aryloxy group, or heterocyclyloxy group. Still other alkyl groups include alkyl groups that have an amine, alkylamine, dialkylamine, arylamine, (alkyl)(aryl)amine, diarylamine, heterocyclylamine, (alkyl)(heterocyclyl)amine, (aryl)(heterocyclyl)amine, or diheterocyclylamine group.

Alkoxy refers to an “-Oalkyl” group, where alkyl is as defined above. A “lower alkoxy” group intends an alkoxy group containing one to six, more preferably one to four, carbon atoms.

Alkenyloxy refers to an “—Oalkenyl” group, wherein alkenyl is as defined above.

Amide refers to —C(O)NR′R″, where R′ and R″ are independently selected from hydrogen, alkyl, aryl, and alkylaryl.

Amine refers to an —N(R′)R″ group, where R′ and R″ are independently selected from hydrogen, alkyl, aryl, and alkylaryl.

Alkylamine refers to a group of the formula —NHR″ and “dialkylamine” refers to a group of the formula —NR″R″, where each R″ is independently an alkyl

Arylamine refers to a group of the formula —NHR″ and “diarylamine” refers to a group of the formula —NR″R″, where each R″ is independently an aryl

Arylamine refers to aromatic groups that have at least one ring having a conjugated pi electron system and includes carbocyclic aryl, heterocyclic aryl and biaryl groups, all of which are substituted with an amine.

Alkylarylamine refers to an alkylamine group substituted with an aryl group.

Aryloxy refers to a group having the formula, R—O—, wherein R is an aryl group

Carbamoyl refers to the group —CONH— which is bonded on one end to the remainder of the molecule and on the other to hydrogen or an organic moiety (such as alkyl, substituted alkyl, aryl, substituted aryl, heterocycle, alkylcarbonyl, hydroxyl and substituted nitrogen).

In one embodiment, an aliphatic functional group is preferably substituted with an aryl group such as an (C6-C12) aryl group mentioned herein below, which may in turn also be substituted, as also described herein. An example of a substituted aryl group includes an “aralkyl group”, which can be substituted or non-substituted.

Accordingly, “aralkyl” refers to unsubstituted alkyl groups as defined above in which a hydrogen or carbon bond of the unsubstituted alkyl group is replaced with a bond to an aryl group as defined above. For example, methyl (—CH₃) is an unsubstituted alkyl group. If a hydrogen atom of the methyl group is replaced by a bond to a phenyl group, such as if the carbon of the methyl were bonded to a carbon of benzene, then the compound is an unsubstituted aralkyl group (i.e., a benzyl group). Thus includes, but is not limited to, groups such as benzyl, diphenylmethyl, and 1-phenylethyl (—CH(C₆H₅)(CH₃)), 2-phenylethyl group, 2-naphthylethyl group, and the like.

“Substituted aralkyl” has the same meaning with respect to unsubstituted aralkyl groups that substituted aryl groups had with respect to unsubstituted aryl groups. However, a substituted aralkyl group also includes groups in which a carbon or hydrogen bond of the alkyl part of the group is replaced by a bond to a non-carbon or a non-hydrogen atom. Examples of substituted aralkyl groups include, but are not limited to, —CH₂C(═O)(C₆H₅), and —CH₂(2-methylphenyl) among others.

In one embodiment, the optionally substituted aliphatic residue comprises or consists of a C₅-C₂₀ alkyl group. Shorter chain lengths can also occur, typically when the alkyl is substituted with an aryl or heteroaryl residue. Further examples of alkyl groups substituted with aryl or heteroaryl includes, for example, a linear (C1-C10), branched (C4-C10) or cyclic (C5-C10) group, such as a methyl group, ethyl group, propyl group, such as a n-propyl group and an isopropyl group, butyl group, such as n-butyl group, isobutyl group, t-butyl group, n-amyl group, pentyl group, such as neopentyl group, cyclopentyl group, hexyl group, such as n-hexyl group, cyclohexyl group, heptyl group, octyl group, such as n-octyl group, nonyl group, such as n-nonyl group, decyl group, such as n-decyl group, undecyl group, dodecyl group, mentyl group, 2,3,4-trimethyl-3-pentyl group, 2,4-dimethyl-3-pentyl group, and the like.

In one embodiment, a C₅-C₂₀ alkyl group can also be substituted, for example with a halogen atom, an alkoxy group, an aryloxy group, an alkylthio group, or an arylthio group. Examples of the halogen atom are a fluorine atom, a chlorine atom, a bromine atom, and an iodine atom. Examples of the alkoxyl group include, for example, a (C1-C4) alkoxy group such as methoxy group, ethoxy group, n-propoxy group, t-butoxy group or the like. Examples of the alkylthio group include, for example, those comprised of the (C1-C10)alkyl group, as described above, and thio group, and specific examples thereof include, for example, n-propylthio group, t-butylthio group or the like. Examples of the arylthio group include, for example, those comprised of the (C6-C12) aryl group, as described above, and a thio group, and specific examples thereof include, for example, a phenylthio group or the like. Examples of the aryloxy group, which may be present on the aryl, heteroaryl, and saturated hydrocarbon groups, for example, those comprised of the (C6-C12) aryl group, as described above, and an oxy group, and specific examples thereof include, for example, a phenoxy group or the like.

The alkyl groups described herein above can contain one or more carbon-carbon double bonds (alkenyl groups) or one or more carbon-carbon triple bonds (alkynyl groups).

“Alkenyl” refers to straight and branched chain and cyclic groups such as those described with respect to unsubstituted alkyl groups as defined above, except that at least one double bond exists between two carbon atoms. Examples include, but are not limited to vinyl, —CH═C(H)(CH₃), —CH═C(CH₃)₂, —C(CH₃)═C(H)₂, —C(CH₃)═C(H)(CH₃), —C(CH₂CH₃)═CH₂, cyclohexenyl, cyclopentenyl, cyclohexadienyl, butadienyl, pentadienyl, and hexadienyl among others.

“Substituted alkenyl” has the same meaning with respect to unsubstituted alkenyl groups that substituted alkyl groups had with respect to unsubstituted alkyl groups. A substituted alkenyl group includes alkenyl groups in which a non-carbon or non-hydrogen atom is bonded to a carbon double bonded to another carbon and those in which one of the non-carbon or non-hydrogen atoms is bonded to a carbon not involved in a double bond to another carbon.

“Alkynyl” refers to straight and branched chain groups such as those described with respect to alkyl groups as defined above, except that at least one triple bond exists between two carbon atoms. Examples include, but are not limited to —CC(H), —CC(CH₃), —CC(CH₂CH₃), —C(H₂)CC(H), —C(H)₂CC(CH₃), and —C(H)₂CC(CH₂CH₃) among others.

“Substituted alkynyl” has the same meaning with respect to unsubstituted alkynyl groups that substituted alkyl groups had with respect to unsubstituted alkyl groups. A substituted alkynyl group includes alkynyl groups in which a non-carbon or non-hydrogen atom is bonded to a carbon triple bonded to another carbon and those in which a non-carbon or non-hydrogen atom is bonded to a carbon not involved in a triple bond to another carbon.

Further examples of substituted alkyl groups are described herein below.

“Alkylaminoalkyl” refers to an unsubstituted alkyl group as defined above in which a carbon or hydrogen bond is replaced by a bond to a nitrogen atom that is bonded to a hydrogen atom and an unsubstituted alkyl group as defined above. For example, methyl (—CH₃) is an unsubstituted alkyl group. If a hydrogen atom of the methyl group is replaced by a bond to a nitrogen atom that is bonded to a hydrogen atom and an ethyl group, then the resulting compound is —CH₂—N(H)(CH₂CH₃) which is an unsubstituted alkylaminoalkyl group.

“Substituted alkylaminoalkyl” refers to an unsubstituted alkylaminoalkyl group as defined above except where one or more bonds to a carbon or hydrogen atom in one or both of the alkyl groups is replaced by a bond to a non-carbon or non-hydrogen atom as described above with respect to substituted alkyl groups except that the bond to the nitrogen atom in all alkylaminoalkyl groups does not by itself qualify all alkylaminoalkyl groups as being substituted. However, substituted alkylaminoalkyl groups does include groups in which the hydrogen bonded to the nitrogen atom of the group is replaced with a non-carbon and non-hydrogen atom.

“Dialkylaminoalkyl” refers to an unsubstituted alkyl group as defined above in which a carbon bond or hydrogen bond is replaced by a bond to a nitrogen atom which is bonded to two other similar or different unsubstituted alkyl groups as defined above.

“Substituted dialkylaminoalkyl” refers to an unsubstituted dialkylaminoalkyl group as defined above in which one or more bonds to a carbon or hydrogen atom in one or more of the alkyl groups is replaced by a bond to a non-carbon and non-hydrogen atom as described with respect to substituted alkyl groups. The bond to the nitrogen atom in all dialkylaminoalkyl groups does not by itself qualify all dialkylaminoalkyl groups as being substituted.

“Heterocyclyloxyalkyl” refers to an unsubstituted alkyl group as defined above in which a carbon bond or hydrogen bond is replaced by a bond to an oxygen atom which is bonded to an unsubstituted heterocyclyl group as defined above.

“Substituted heterocyclyloxyalkyl” refers to an unsubstituted heterocyclyloxyalkyl group as defined above in which a bond to a carbon or hydrogen group of the alkyl group of the heterocyclyloxyalkyl group is bonded to a non-carbon and non-hydrogen atom as described above with respect to substituted alkyl groups or in which the heterocyclyl group of the heterocyclyloxyalkyl group is a substituted heterocyclyl group as defined above.

“Arylaminoalkyl” refers to an unsubstituted alkyl group as defined above in which a carbon bond or hydrogen bond is replaced by a bond to a nitrogen atom which is bonded to at least one unsubstituted aryl group as defined above.

“Substituted arylaminoalkyl” refers to an unsubstituted arylaminoalkyl group as defined above except where either the alkyl group of the arylaminoalkyl group is a substituted alkyl group as defined above or the aryl group of the arylaminoalkyl group is a substituted aryl group except that the bonds to the nitrogen atom in all arylaminoalkyl groups does not by itself qualify all arylaminoalkyl groups as being substituted. However, substituted arylaminoalkyl groups does include groups in which the hydrogen bonded to the nitrogen atom of the group is replaced with a non-carbon and non-hydrogen atom.

“Heterocyclylaminoalkyl” refers to an unsubstituted alkyl group as defined above in which a carbon or hydrogen bond is replaced by a bond to a nitrogen atom which is bonded to at least one unsubstituted heterocyclyl group as defined above.

“Substituted heterocyclylaminoalkyl” refers to unsubstituted heterocyclylaminoalkyl groups as defined above in which the heterocyclyl group is a substituted heterocyclyl group as defined above and/or the alkyl group is a substituted alkyl group as defined above. The bonds to the nitrogen atom in all heterocyclylaminoalkyl groups does not by itself qualify all heterocyclylaminoalkyl groups as being substituted. However, substituted heterocyclylaminoalkyl groups do include groups in which the hydrogen bonded to the nitrogen atom of the group is replaced with a non-carbon and non-hydrogen atom.

“Alkylaminoalkoxy” refers to an unsubstituted alkyl group as defined above in which a carbon or hydrogen bond is replaced by a bond to an oxygen atom which is bonded to the parent compound and in which another carbon or hydrogen bond of the unsubstituted alkyl group is bonded to a nitrogen atom which is bonded to a hydrogen atom and an unsubstituted alkyl group as defined above.

“Substituted alkylaminoalkoxy” refers to unsubstituted alkylaminoalkoxy groups as defined above in which a bond to a carbon or hydrogen atom of the alkyl group bonded to the oxygen atom which is bonded to the parent compound is replaced by one or more bonds to a non-carbon and non-hydrogen atoms as discussed above with respect to substituted alkyl groups and/or if the hydrogen bonded to the amino group is bonded to a non-carbon and non-hydrogen atom and/or if the alkyl group bonded to the nitrogen of the amine is bonded to a non-carbon and non-hydrogen atom as described above with respect to substituted alkyl groups. The presence of the amine and alkoxy functionality in all alkylaminoalkoxy groups does not by itself qualify all such groups as substituted alkylaminoalkoxy groups.

“Unsubstituted dialkylaminoalkoxy” refers to an unsubstituted alkyl group as defined above in which a carbon or hydrogen bond is replaced by a bond to an oxygen atom which is bonded to the parent compound and in which another carbon or hydrogen bond of the unsubstituted alkyl group is bonded to a nitrogen atom which is bonded to two other similar or different unsubstituted alkyl groups as defined above.

“Substituted dialkylaminoalkoxy” refers to an unsubstituted dialkylaminoalkoxy group as defined above in which a bond to a carbon or hydrogen atom of the alkyl group bonded to the oxygen atom which is bonded to the parent compound is replaced by one or more bonds to a non-carbon and non-hydrogen atoms as discussed above with respect to substituted alkyl groups and/or if one or more of the alkyl groups bonded to the nitrogen of the amine is bonded to a non-carbon and non-hydrogen atom as described above with respect to substituted alkyl groups. The presence of the amine and alkoxy functionality in all dialkylaminoalkoxy groups does not by itself qualify all such groups as substituted dialkylaminoalkoxy groups.

“Heterocyclyloxy” refers to a hydroxyl group (—OH) in which the bond to the hydrogen atom is replaced by a bond to a ring atom of an otherwise unsubstituted heterocyclyl group as defined above.

“Substituted heterocyclyloxy” refers to a hydroxyl group (—OH) in which the bond to the hydrogen atom is replaced by a bond to a ring atom of an otherwise substituted heterocyclyl group as defined above.

Aromatic Residues

Aromatic residues can be optionally substituted aryl or heteroaryl residues. Aryl includes, but is not limited to, groups such as phenyl, biphenyl, anthracenyl, naphthenyl by way of example. Although aryl includes groups containing condensed rings such as naphthalene, it does not include aryl groups that have other groups such as alkyl or halo groups bonded to one of the ring members, as aryl groups such as tolyl are considered herein to be substituted aryl groups as described herein below. Aryl groups may be bonded to one or more carbon atom(s), oxygen atom(s), nitrogen atom(s), and/or sulfur atom(s) in the ligand.

Substituted aryl group has the same meaning with respect to unsubstituted aryl groups that substituted alkyl groups had with respect to unsubstituted alkyl groups. However, a substituted aryl group also includes aryl groups in which one of the aromatic carbons is bonded to one of the non-carbon or non-hydrogen atoms described above and also includes aryl groups in which one or more aromatic carbons of the aryl group is bonded to a substituted and/or unsubstituted alkyl, alkenyl, or alkynyl group as defined herein. This includes bonding arrangements in which two carbon atoms of an aryl group are bonded to two atoms of an alkyl, alkenyl, or alkynyl group to define a fused ring system (e.g. dihydronaphthyl or tetrahydronaphthyl).

Examples of aryl and heteroaryl include, for example, a (C6-C12) aryl group such as a phenyl group, tolyl group, naphthyl group, biphenyl group or the like, and a (C4-C5) heteroaryl group, or pyridyl group, or the like.

Also, when the at least one functional group comprises or consists of an optionally substituted aromatic residue, the aromatic residue can be selected from the group consisting of aromatic residues comprising or consisting of fluorenyl, pyrroyl, furanyl, thienyl, thiophenyl, thiazolyl, isoindolyl, quinolinyl, isoquinolinyl, oxazolyl, and purinyl. Further examples include, but is not limited to tetrahydrothiophenyl, sulfur oxidized tetrahydrothiophenyl, tetrazolyl, benzofuranyl, thianaphthalenyl, indolenyl, benzimidazolyl, piperidinyl, 4-piperidonyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thianthrenyl, pyranyl, isobenzofuranyl, chromenyl, xanthenyl, phenoxathiinyl, 2H-pyrrolyl, isothiazolyl, isoxazolyl, pyrazinyl, pyridazinyl, indolizinyl, isoindolyl, 3H-indolyl, 1H-indazoly, 4H-quinolizinyl, phthalazinyl, naphthyridinyl, quinoxalinyl, quinazolinyl, cinnolinyl, pteridinyl, 4aH-carbazolyl, carbazolyl, beta-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, quinuclidinyl, morpholinyl, oxazolidinyl, benzotriazolyl, benzisoxazolyl, oxindolyl, benzoxazolinyl, and isatinoyl.

By way of example and not limitation, carbon bonded heterocycles can be bonded at position 176, 3, 4, 5, or 6 of a pyridine, position 3, 4, 5, or 6 of a pyridazine, position 176, 4, 5, or 6 of a pyrimidine, position 176, 3, 5, or 6 of a pyrazine, position 176, 3, 4, or 5 of a furan, tetrahydrofuran, thiofuran, thiophene, pyrrole or tetrahydropyrrole, position 176, 4, or 5 of an oxazole, imidazole or thiazole, position 3, 4, or 5 of an isoxazole, pyrazole, or isothiazole, position 2 or 3 of an aziridine, position 176, 3, or 4 of an azetidine, position 176, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or beta-carboline. Typically, nitrogen bonded heterocycles include 1-aziridyl, 1-azetedyl, 1-pyrrolyl, 1-imidazolyl, 1-pyrazolyl, and 1-piperidinyl.

As will be clear from the above, the heteroaromatic group can also be selected from the group consisting of heteroaromatic groups comprising or consisting of optionally substituted, fused heteroaromatic compounds. Examples include e.g. indole, benzothiophene, benzotriazene and quinoline.

In one embodiment, the aromatic residue can be substituted with one or more optionally substituted aliphatic groups, such as the optionally substituted aliphatic groups mentioned herein immediately above, such as the linear, branched or cyclic (C1-C10)alkyl group, for example a methyl group, an ethyl group, an isopropyl group, a n-butyl group, a t-butyl group, a n-amyl group, a n-hexyl group and the like.

The aromatic residue can be substituted with one or more heteroatoms, or substituted with one or more aromatic groups, or substituted with one or more heteroaromatic groups.

Also, the aromatic residue can e.g. be substituted with a substituted alkyl or aryl or heteroaryl, wherein the aromatic residue, or the alkyl or the aryl or the heteroaryl is substituted with a heteroatom selected from O, N, S and halogen, or substituted with one or more groups selected from hydroxyl, amino, thiol, halogen, carbonyl, carboxylic acid, ether and ester.

The aryl and heteroaryl groups can also be substituted, for example, with a halogen atom, an alkoxy group, an aryloxy group, an alkylthio group, or an arylthio group. Examples of the halogen atom are a fluorine atom, a chlorine atom, a bromine atom, and an iodine atom. Examples of the alkoxyl group include, for example, a (C1-C4) alkoxy group such as methoxy group, ethoxy group, n-propoxy group, t-butoxy group or the like. Examples of the alkylthio group include, for example, those comprised of the (C1-C10)alkyl group, as described above, and thio group, and specific examples thereof include, for example, n-propylthio group, t-butylthio group or the like. Examples of the arylthio group include, for example, those comprised of the (C6-C12) aryl group, as described above, and a thio group, and specific examples thereof include, for example, a phenylthio group or the like. Examples of the aryloxy group, which may be present on the aryl, heteroaryl, and saturated hydrocarbon groups, for example, those comprised of the (C6-C12) aryl group, as described above, and an oxy group, and specific examples thereof include, for example, a phenoxy group or the like.

The term heterocyclyl as used herein refers to both aromatic and nonaromatic ring compounds including monocyclic, bicyclic, and polycyclic ring compounds such as, but not limited to, quinuclidyl, containing 3 or more ring members of which one or more is a heteroatom such as, but not limited to, N, O, and S. Although heterocyclyl includes condensed heterocyclic rings such as benzimidazolyl, it does not include heterocyclyl groups that have other groups such as alkyl or halo groups bonded to one of the ring members as compounds such as 2-methylbenzimidazolyl are substituted heterocyclyl groups. Examples of heterocyclyl groups include, but are not limited to: unsaturated 3 to 8 membered rings containing 1 to 4 nitrogen atoms such as, but not limited to pyrrolyl, pyrrolinyl, imidazolyl, pyrazolyl, pyridyl, dihydropyridyl, pyrimidyl, pyrazinyl, pyridazinyl, triazolyl (e.g. 4H-1,2,4-triazolyl, 1H-1,2,3-triazolyl, 2H-1,2,3-triazolyl etc.), tetrazolyl, (e.g. 1H-tetrazolyl, 2H tetrazolyl, etc.); saturated 3 to 8 membered rings containing 1 to 4 nitrogen atoms such as, but not limited to, pyrrolidinyl, imidazolidinyl, piperidinyl, piperazinyl; condensed unsaturated heterocyclic groups containing 1 to 4 nitrogen atoms such as, but not limited to, indolyl, isoindolyl, indolinyl, indolizinyl, benzimidazolyl, quinolyl, isoquinolyl, indazolyl, benzotriazolyl; unsaturated 3 to 8 membered rings containing 1 to 2 oxygen atoms and 1 to 3 nitrogen atoms such as, but not limited to, oxazolyl, isoxazolyl, oxadiazolyl (e.g. 1,2,4-oxadiazolyl, 1,3,4-oxadiazolyl, 1,2,5-oxadiazolyl, etc.); saturated 3 to 8 membered rings containing 1 to 2 oxygen atoms and 1 to 3 nitrogen atoms such as, but not limited to, morpholinyl; unsaturated condensed heterocyclic groups containing 1 to 2 oxygen atoms and 1 to 3 nitrogen atoms, for example, benzoxazolyl, benzoxadiazolyl, benzoxazinyl (e.g. 2H-1,4-benzoxazinyl etc.); unsaturated 3 to 8 membered rings containing 1 to 3 sulfur atoms and 1 to 3 nitrogen atoms such as, but not limited to, thiazolyl, isothiazolyl, thiadiazolyl (e.g. 1,2,3-thiadiazolyl, 1,2,4-thiadiazolyl, 1,3,4-thiadiazolyl, 1,2,5-thiadiazolyl, etc.); saturated 3 to 8 membered rings containing 1 to 2 sulfur atoms and 1 to 3 nitrogen atoms such as, but not limited to, thiazolodinyl; saturated and unsaturated 3 to 8 membered rings containing 1 to 2 sulfur atoms such as, but not limited to, thienyl, dihydrodithiinyl, dihydrodithionyl, tetrahydrothiophene, tetrahydrothiopyran; unsaturated condensed heterocyclic rings containing 1 to 2 sulfur atoms and 1 to 3 nitrogen atoms such as, but not limited to, benzothiazolyl, benzothiadiazolyl, benzothiazinyl (e.g. 2H-1,4-benzothiazinyl, etc.), dihydrobenzothiazinyl (e.g. 2H-3,4dihydrobenzothiazinyl, etc.), unsaturated 3 to 8 membered rings containing oxygen atoms such as, but not limited to furyl; unsaturated condensed heterocyclic rings containing 1 to 2 oxygen atoms such as benzodioxolyl (e.g. 1,3-benzodioxoyl, etc.); unsaturated 3 to 8 membered rings containing an oxygen atom and 1 to 2 sulfur atoms such as, but not limited to, dihydrooxathiinyl; saturated 3 to 8 membered rings containing 1 to 2 oxygen atoms and 1 to 2 sulfur atoms such as 1,4-oxathiane; unsaturated condensed rings containing 1 to 2 sulfur atoms such as benzothienyl, benzodithiinyl; and unsaturated condensed heterocyclic rings containing an oxygen atom and 1 to 2 oxygen atoms such as benzoxathiinyl. Heterocyclyl group also include those described above in which one or more S atoms in the ring is double-bonded to one or two oxygen atoms (sulfoxides and sulfones). For example, heterocyclyl groups include tetrahydrothiophene, tetrahydrothiophene oxide, and tetrahydrothiophene 1,1-dioxide. Preferred heterocyclyl groups contain 5 or 6 ring members. More preferred heterocyclyl groups include morpholine, piperazine, piperidine, pyrrolidine, imidazole, pyrazole, 1,2,3-triazole, 1,2,4triazole, tetrazole, thiomorpholine, thiomorpholine in which the S atom of the thiomorpholine is bonded to one or more O atoms, pyrrole, homopiperazine, oxazolidin-2-one, pyrrolidin-2-one, oxazole, quinuclidine, thiazole, isoxazole, furan, and tetrahydrofuran.

Substituted heterocyclyl refers to an unsubstituted heterocyclyl group as defined above in which one of the ring members is bonded to a non-hydrogen atom such as described above with respect to substituted alkyl groups and substituted aryl groups. Examples, include, but are not limited to, 2-methylbenzimidazolyl, 5-methylbenzimidazolyl, 5-chlorobenzthiazolyl, 1-methyl piperazinyl, and 2-chloropyridyl among others.

Heterocyclylalkyl refers to unsubstituted alkyl groups as defined above in which a hydrogen or carbon bond of the unsubstituted alkyl group is replaced with a bond to a heterocyclyl group as defined above. For example, methyl (—CH₃) is an unsubstituted alkyl group. If a hydrogen atom of the methyl group is replaced by a bond to a heterocyclyl group, such as if the carbon of the methyl were bonded to carbon 2 of pyridine (one of the carbons bonded to the N of the pyridine) or carbons 3 or 4 of the pyridine, then the compound is an unsubstituted heterocyclylalkyl group.

Substituted heterocyclylalkyl has the same meaning with respect to unsubstituted heterocyclylalkyl groups that substituted aralkyl groups had with respect to unsubstituted aralkyl groups. However, a substituted heterocyclylalkyl group also includes groups in which a non-hydrogen atom is bonded to a heteroatom in the heterocyclyl group of the heterocyclylalkyl group such as, but not limited to, a nitrogen atom in the piperidine ring of a piperidinylalkyl group.

Manufacturing the Compounds of the Present Invention

As known to one skilled in the art, many methods are available for manufacturing the compounds of the present invention, such as for example using either solid or liquid phase methods described in standard textbooks, or for example by a combination of both methods. Starting materials or indeed the compounds themselves may be commercially available from chemical vendors such as Aldrich, Sigma, Nova Biochemicals, Bachem Biosciences, and the like, or may be readily synthesized by known procedures, for example using e.g. production steps similar to those described in WO 02/069970 (Merck and Co.), WO 01/90091 (Biovitrum AB), FR 2675801 (Rhone-Poulenc Rorer), U.S. Pat. No. 4,714,700 (Fournier et al., ), WO 97/29097 (Smithkline Beecham PLC.) WO 97/48681 (Smithkline Beecham PLC), WO 00/56712 (Smithkline Beecham PLC), WO 2005/011686 (Applied Research Systems ARS Holding) as well as references cited by Pouzet (“SB-258741: A 5-HT7 Receptor Antagonist of Potential Clinical Interest”, CNS Drug reviews, Vol. 8, No. 1, p 90-100).

Some of the compounds can also be obtained from a commercial supplier, such as e.g. EMD Biosciences (http://www.emdbiosciences.com, supplying e.g. 1-NSC).

The compounds are optionally further purified/formulated for pharmaceutical use, as is known to one skilled in the art.

Uses of the Compounds According to the Present Invention

In one aspect of the present invention, the compounds provided herein can be used as a medicament. The compounds of the invention are believed to act through SERT, preferably by having an affinity to SERT.

However, the compounds provided herein can also be used as a medicament acting through DAT, and/or NET.

In one embodiment of the present invention, the compounds disclosed herein are useful in the prophylaxis and/or treatment of a CNS disorder, such as: Parkinson's disease, Huntington's disease, anxiety, depression, manic depression, psychosis, epilepsy, obsessive compulsive disorders, migraine, Alzheimer's disease (enhancement of cognitive memory), sleep disorders, feeding disorders such as anorexia and bulimia, panic attacks, attention deficit hyperactivity disorder (ADHD), attention deficit disorder (ADD), schizophrenia, disorders associated with spinal trauma and/or head injury such as hydrocephalus, and withdrawal from drug abuse such as cocaine, ethanol, nicotine and benzodiazepines.

In another embodiment of the present invention, the compounds disclosed herein are useful in the prophylaxis and/or treatment of GI (gastrointestinal) disorders, such as functional bowel disorder or irritable bowel syndrome (IBS), as well as for the treatment of obesity.

In another embodiment of the present invention, the compounds disclosed herein are useful in the prophylaxis and/or treatment of stroke or a neurodegenerative disease, such as Alzheimer's disease, multi-infarct dementia, AIDS dementia, Huntington's disease, Parkinson's disease, amyotrophic lateral sclerosis or other conditions in which neuronal cell death occurs, such as stroke or head trauma.

In another embodiment of the present invention, the compounds disclosed herein are useful in the prophylaxis and/or treatment of neurological and neuropsychiatric disorders, including psychoses such as schizophrenia, dementia and other forms of impaired cognition such as attention deficit disorders and organic brain syndromes, drug-induced (phencyclidine, ketamine and other dissociative anaesthetics, amphetamine and other psychostimulants and cocaine) psychosis, psychosis associated with affective disorders, brief reactive psychosis, schizoaffective psychosis, and psychosis NOS, “schizophreniaspectrum” disorders such as schizoid or schizotypal personality disorders, or illness associated with psychosis (such as major depression, manic depressive (bipolar) disorder, Alzheimer's disease and post-traumatic stress syndrome), and NMDA receptor related disorders such as autism, depression, benign forgetfulness, childhood learning disorders and closed head injury.

In another embodiment of the present invention, the compounds disclosed herein are useful in the prophylaxis and/or treatment of neural disorders. By “neural disorders” herein is meant disorders of the central and/or peripheral nervous system that are associated with neuron degeneration or damage. Specific examples of neural disorders include, but are not limited to, Alzheimer's disease, Parkinson's disease, Huntington's chorea, stroke, ALS, peripheral neuropathies, and other conditions characterized by necrosis or loss of neurons, whether central, peripheral, or motor neurons, in addition to treating damaged nerves due to trauma, burns, kidney dysfunction or injury, pancreatic dysfunction or injury, lung dysfunction or injury, injury to fatty tissue, and the toxic effects of chemotherapeutics used to treat cancer and AIDS. For example, peripheral neuropathies associated with certain conditions, such as neuropathies associated with diabetes, AIDS, or chemotherapy may be treated using the formulations of the present invention.

In various embodiments of the invention, agents are administered to patients in whom the nervous system has been damaged by trauma, surgery, stroke, ischemia, infection, metabolic disease, nutritional deficiency, malignancy, or toxic agents, to promote the survival or growth of neurons, or in whatever conditions are treatable with NGF, NT-3, BDNF or NT4-5. For example, agents of the invention can be used to promote the survival or growth of motor neurons that are damaged by trauma or surgery. Also, agents of the invention can be used to treat motoneuron disorders, such as amyotrophic lateral sclerosis (Lou Gehrig's disease), Bell's palsy, and various conditions involving spinal muscular atrophy, or paralysis. Agents of the present invention can be used to treat human neurodegenerative disorders, such as Alzheimer's disease, Parkinson's disease, epilepsy, multiple sclerosis, Huntington's chorea, Down's Syndrome, nerve deafness, and Meniere's disease. Agents of the present invention can be used as cognitive enhancer, to enhance learning particularly in dementias or trauma.

Further, agents of the present invention are preferably used to treat neuropathy, and especially peripheral neuropathy. “Peripheral neuropathy” refers to a disorder affecting the peripheral nervous system, most often manifested as one or a combination of motor, sensory, sensorimotor, or autonomic neural dysfunction. The wide variety of morphologies exhibited by peripheral neuropathies can each be attributed uniquely to an equally wide number of causes. For example, peripheral neuropathies can be genetically acquired, can result from a systemic disease, or can be induced by a toxic agent. Examples include, but are not limited to, diabetic peripheral neuropathy, distal sensorimotor neuropathy, or autonomic neuropathies such as reduced motility of the gastrointestinal tract or atony of the urinary bladder. Examples of neuropathies associated with systemic disease include post-polio syndrome or AIDS-associated neuropathy; examples of hereditary neuropathies include Charcot-Marie-Tooth disease, Refsum's disease, Abetalipoproteinemia, Tangier disease, Krabbe's disease, Metachromatic leukodystrophy, Down's Syndrome, Fabry's disease, and Dejerine-Sottas syndrome; and examples of neuropathies caused by a toxic agent include those caused by treatment with a chemotherapeutic agent such as vincristine, cisplatin, methotrexate, or 3′-azido-3′-deoxythymidine. Other neural diseases that could benefit from treatment with one or more agents of the present invention include depression and mania.

In another embodiment of the present invention, the compounds disclosed herein are useful in the prophylaxis and/or treatment of a CNS disorder, such as anxiety, depression, sleep disorders, migraine, Parkinson's disease, schizophrenia, pain, appetite disorders and other indications such as inflammation, spastic colon, renal disorders, hypotension cardiovascular shock, septic shock or gastrointestinal diseases. In different embodiments, the CNS disorder is an eating disorder, obesity, a psychotic disorder, anxiety, a learning disorder, a memory disorder, an electrolyte balance disorder, diuresis, diabetes, an intestinal motility disorder, irritable bowel syndrome, nicotine addiction, or a cardiovascular disorder. In different embodiments, the abnormality is a lower urinary tract disorder, interstitial cystitis, a steroid hormone disorder, an epinephrine release disorder, a gastrointestinal disorder, hypoglycemia, a respiratory disorder, asthma, a reproductive function disorder, an immune disorder, an endocrine disorder, a musculoskeletal disorder, a neuroendocrine disorder, a cognitive disorder, a sensory modulation and transmission disorder, a motor coordination disorder, a sensory integration disorder, a motor integration disorder, a dopaminergic function disorder, an appetite disorder, a serotonergic function disorder, an olfaction disorder, nasal congestion, a sympathetic innervation disorder, an affective disorder, morphine tolerance, opiate addiction, or migraine.

In one preferred embodiment of the present invention, the compounds disclosed herein are useful for treatment or prophylaxis of depression.

In another embodiment of the present invention, the compounds disclosed herein are useful for pain relief.

In yet another embodiment of the present invention, the compounds disclosed herein are useful for the treatment or prophylaxis of drug abuse, for example cocain abuse.

In one preferred embodiment of the present invention, the compounds disclosed herein are useful for treatment or prophylaxis of a disorders selected from the group consisting of: depression, migraine pain, bulimia, premenstrual syndrome or late luteal phase syndrome, alcoholism, tobacco abuse, panic disorder, anxiety, general pain, chronic pain, post-traumatic syndrome, memory loss, dementia of aging, social phobia, attention deficit hyperactivity disorder, disruptive behavior disorders, impulse control disorders, borderline personality disorder, obsessive compulsive disorder, chronic fatigue syndrome, premature ejaculation, erectile difficulty, anorexia nervosa, disorders of sleep, autism, mutism, allergic rhinitis, trichotillomania, trigeminal neuralgia, dental pain or temperomandibular joint dysfunction pain.

Thus, the compounds of the present invention can be used for the manufacture of a medicament for the treatment or prophylaxis of a CNS disorder. Preferably, said CNS disorder is selected from the group consisting of: anxiety, social anxiety disorder, generalized anxiety disorder, post-traumatic stress disorder, panic disorder, a sleep disorder, an eating disorder, or obsessive compulsive disorder (OCD). In another preferred embodiment, said CNS disorder is a psychiatric disease, such as selected from the group consisting of depression, anxiety, mania, ecstasy syndrome, obsessive compulsory disorder (OCD) and/or eating disorders. Thus, the compounds of the present invention can be used for prophylaxis or treatment of depression.

Formulation

In general, compounds of the present invention will be administered as pharmaceutical formulations including those suitable for oral (including buccal and sublingual), rectal, nasal, topical, pulmonary, vaginal, or parenteral (including intramuscular, intraarterial, intrathecal, subcutaneous and intravenous) administration or in a form suitable for administration by inhalation or insufflation. The preferred manner of administration is generally oral using a convenient daily dosage regimen which can be adjusted according to the degree of affliction.

To prepare the pharmaceutical compositions of this invention, an appropriate amount of the active ingredient (s), in salt form or base form, is combined in an intimate admixture with a pharmaceutically acceptable carrier, which can take a wide variety of forms depending on the form of preparation desired for administration. These pharmaceutical compositions are desirably in unitary dosage form suitable for administration orally, rectally, percutaneously or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed.

It is especially advantageous to formulate the aforementioned pharmaceutical compositions in dosage unit form for ease of administration and uniformity of dosage. As used in the specification and claims, unit dosage form refers to physically discrete units suitable as unitary dosages, each unit containing a predetermined quantity of active ingredient (s) calculated to produce the desired therapeutic effect, in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

A compound or compounds of the present invention, together with one or more conventional adjuvants, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. For example, in one embodiment, formulations containing about one (1) milligram of active ingredient or, more broadly, about 0.01 to about one hundred (100) milligrams, per tablet, are suitable unit dosage forms.

The compounds of the present invention may be formulated in a wide variety of oral administration dosage forms. The pharmaceutical compositions and dosage forms may comprise a compound or compounds of the present invention or pharmaceutically acceptable salts thereof as the active component. The pharmaceutically acceptable carriers may be either solid or liquid. Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier, providing a capsule in which the active component, with or without carriers, is surrounded by a carrier, which is in association with it. Similarly, cachets and lozenges are included. Tablets, powders, capsules, pills, cachets, and lozenges may be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

Solid form preparations include solutions, suspensions, and emulsions, and may contain, in addition to the active component, colorants, flavors, stabilizers, buffers, artificial and natural sweeteners, dispersants, thickeners, solubilizing agents, and the like.

The compounds of the present invention may be formulated for parenteral administration (e. g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e. g., olive oil), and injectable organic esters (e. g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e. g., sterile, pyrogen-free water.

The compounds of the present invention may be formulated for topical administration to the epidermis as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatine and glycerine or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa buffer is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve.

Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP).

The powder carrier can for example form a gel in the nasal cavity. The powder composition may be presented in unit dose form for example in capsules or cartridges of e. g., gelatine or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to a skin-adhesive solid support. The compound of interest can also be combined with a penetration enhancer, e. g., Azone (1-dodecylazacycloheptan-2-one). Sustained release delivery systems are inserted subcutaneously into the subdermal layer by surgery or injection. The subdermal implants encapsulate the compound in a lipid soluble membrane, e. g., silicone rubber, or a biodegradable polymer, e. g., polylactic acid.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Other suitable pharmaceutical carriers and their formulations are described in Remington: The Science and Practice of Pharmacy 1995, edited by E. W. Martin, Mack Publishing Company,19th edition, Easton, Pa.

Administration

For administration, the compounds of this invention are ordinarily combined with one or more adjuvants appropriate for the indicated route of administration.

The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the compounds of this invention may be dissolved in saline, water, polyethylene glycol, propylene glycol, ethanol, corn oil, peanut oil, cottonseed oil, sesame oil, tragacanth gum, benzyl alcohol, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The pharmaceutical compositions may be made up in a solid form including granules, powders or suppositories or in a liquid form such as solutions, suspensions, or emulsions. The pharmaceutical compositions may be subjected to conventional pharmaceutical operations such as sterilization and/or may contain conventional adjuvants, such as preservatives, stabilizers, wetting agents, emulsifiers, buffers, etc.

Solid dosage forms for oral administration may include capsules, tablets, pills, powders, and granules.

In such solid dosage forms, the active compound may be admixed with at least one inert diluent such as sucrose lactose or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water.

Such compositions may also comprise adjuvants, such as wetting agents, emulsifying and suspending agents, and sweetening, flavoring, and perfuming agents.

Compounds of the present invention can possess one or more asymmetric carbon atoms and are thus capable of existing in the form of optical isomers as well as in the form of racemic or non-racemic mixtures thereof.

The optical isomers can be obtained by resolution of the racemic mixtures according to conventional processes, for example by formation of diastereoisomeric salts by treatment with an optically active acid or base.

Examples of appropriate acids are tartaric, diacetyltartaric, dibenzoyltartaric, ditoluoyltartaric and camphorsulfonic acid and then separation of the mixture of diastereoisomers by crystallization followed by liberation of the optically active bases from these salts. A different process for separation of optical isomers involves the use of a chiral chromatography column optimally chosen to maximize the separation of the enantiomers. Still another available method involves synthesis of covalent diastereoisomeric molecules by reacting compounds of this invention with an optically pure acid in an activated form or an optically pure isocyanate. The synthesized diastereoisomers can be separated by conventional means such as chromatography, distillation, crystallization or sublimation, and then hydrolyzed to deliver the enantiomerically pure compound. The optically active compounds of this invention can likewise be obtained by utilizing optically active starting materials. These isomers may be in the form of a free acid, a free base, an ester or a salt.

The compounds of the present invention can be used in the form of salts derived from inorganic or organic acids. These salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate,ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, methanesulfonate, nicotinate, 2-naphthalenesulfonate, oxalate, palmoate, pectinate, persulfate, 3-phenylpropionate, picrate, pivalate, propionate, succinate, tartrate, thiocyanate, tosylate, mesylate and undecanoate. Also, the basic nitrogencontaining groups can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained.

Examples of acids which may be employed to form pharmaceutically acceptable acid addition salts include such inorganic acids as hydrochloric acid, sulphuric acid and phosphoric acid and such organic acids as oxalic acid, maleic acid, succinic acid and citric acid.

Other examples include salts with alkali metals or alkaline earth metals, such as sodium, potassium, calcium or magnesium or with organic bases.

While the compounds of the invention can be administered as the sole active pharmaceutical agent, they can also be used in combination with one or more other agents. When administered as a combination, the therapeutic agents can be formulated as separate compositions which are given at the same time or different times, or the therapeutic agents can be given as a single composition. For example, the compounds according to the present invention may be administered before, during or after the administration of the serotonin reuptake inhibitor, provided that the time between the administration of said compounds and the administration of the serotonin reuptake inhibitor is such that ingredients are allowed to act synergistically on the CNS. When simultaneous administration of the compounds according to the present invention and a serotonin reuptake inhibitor is envisaged, a composition containing both a serotonin reuptake inhibitor and the compounds according to the present invention may be particularly convenient. Alternatively, the compounds according to the present invention and the serotonin reuptake inhibitor may be administered separately in the form of suitable compositions. The compositions may be prepared as described elsewhere herein.

Dosage

In general, the compounds of the present invention will be administered in a therapeutically effective amount by any of the accepted modes of administration for agents that serve similar utilities. In one preferred embodiment of the present invention, the invention comprises a method for treating a disease or disorder in an individual. Said method comprises administering to said individual, in a pharmaceutically acceptable carrier, a sufficient amount of any of the compounds disclosed herein. By “sufficient amount” herein is meant a dose that produces the therapeutic effects for which it is administered. The exact dose will depend on the disorder to be treated, and will be ascertainable by one skilled in the art using known techniques. For example, the compound of the present invention can be administered to an animal in an amount of from 1 μg/kg to about 100 mg/kg per day. In addition, as is known in the art, adjustments for age as well as the body weight, general health, sex, diet, time of administration, drug interaction and the severity of the disease may be necessary, and will be ascertainable with routine experimentation by those skilled in the art.

In another embodiment, suitable dosage ranges are typically 1-500 mg daily, preferably 1-100 mg daily, and most preferably 1-30 mg daily, 30-70 mg daily or about 50 mg daily, depending upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this Application, to ascertain a therapeutically effective amount of the compounds of the present invention for a given disease. For 1-NSC, a dosage of about 100 mg/daily is preferred, optionally split into a number of doses per day, for example two (such as a.m. and p.m. doses). For example, 1-NSC can be dissolved in 30% of (2-hydroxypropyl)-beta-cyclodextrin, (pH6-7 with a few drops of NaOH 1N).

Combination Treatments

It is further envisaged that the compounds of the present invention may be used in combination with at least one other compound. By administration “in combination” is meant herein that said other therapeutic compound may be administered prior to and/or during (including in a co-formulation) and/or after treatment with the compounds of the present invention. In one preferred embodiment, the compounds of the present invention are administered together with one or more other compounds in a “kit-of-parts” system, for simultaneous, sequential or separate administration.

Thus, one aspect of the present invention relates to the combination of a compound according to the present invention with another medicament for a disorder, such as a CNS disorder. Preferably, said other medicament is either co-formulated with said compound according to the present invention, and/or is manufactured as a “kit-of-parts”.

As described above, in one embodiment of the present invention, said other medicament is an anti-depressant, such as a selective serotonin reuptake inhibitor (SSRI). Preferred SSR1 antidepressants include, but are not restricted to, Fluoxetine (Prozac), Fluvoxamine (Luvox), Paroxetine (Paxil, Paxil CR), Sertraline (Zoloft), Citalopram (Celexa) and Escitalopram oxalate (Lexapro).

In another embodiment of the present invention, said other medicament is an anti-depressant, such as a tricyclic antidepressant (TCA). Preferred TCAs include, but are not restricted to, Antidepressant drugs in the tricyclic drug group include: amitriptyline (Elavil®, Endep® Tryptanol®, Lentizol®), amoxapine (Asendin®), clomipramine (Anafranil®), desipramine (Norpramin®, Pertofrane®), dothiepin hydrochloride (Prothiaden®, Thaden®), doxepin (Adapin®, Sinequan®), imipramine (Tofranil®), lofepramine (Gamanil®, Lomont®), nortriptyline (Pamelor®, Allegron®), protriptyline (Vivactil®, Concordin®), trimipramine (Surmontil®).

Many antidepressants with serotonin reuptake inhibiting effect have been described in the literature. Any pharmacologically active compound which primarily or partly exerts its therapeutic effect via inhibition of serotonin reuptake in the CNS, may benefit from augmentation with the compounds of the present invention.

The following list contains a number of serotonin reuptake inhibitors, which may benefit from combination with compounds of the present invention: citalopram, escitalopram, fluoxetine, R-fluoxetine, sertraline, paroxetine, fluvoxamine, venlafaxine, duloxetine, dapoxetine, nefazodone, imipramine, imipramine N-oxide, desipramine, pirandamine, dazepinil, nefopam, befuraline, fezolamine, femoxetine, clomipramine, cianoimipramine, litoxetine, cericlamine, seproxetine, WY 27587, WY 27866, imeldine, ifoxetine, tiflucarbine, viqualine, milnacipran,bazinaprine, YM 922, S 33005, F 98214-TA, OPC 14523, alaproclate, cyanodothepine, trimipramine, quinupramine, dothiepin, Loxapine, nitroxazepine, McN 5652, McN 5707, OI 77, Org 6582, Org 6997, Org 6906, amitriptyline, amitriptyline N-oxide,nortriptyline, CL 255.663, pirlindole, indatraline, LY 113.821, LY 214.281, CGP 6085 A, RU 25.591, napamezole, diclofensine, trazodone, EMD 68.843, BMY 42.569, NS 2389, sercloremine, nitroquipazine, ademethionine, sibutramine and clovoxamine. The compounds mentioned above may be used in the form of the base or a pharmaceutically acceptable acid addition salt thereof. Each of the serotonin reuptake inhibitors specified above is intended to be an individual embodiment. Accordingly, each of them and the use thereof may be claimed individually.

Typically, compounds such as citalopram, escitalopram, fluoxetine, R-fluoxetine, sertraline, paroxetine, fluvoxamine, venlafaxine, desmethylvenlafaxine, duloxetine, dapoxetine, vilazodone, nefazodone, imipramine, imipramine N-oxide, desipramine, pirandamine, dazepinil, nefopam, befuraline,fezolamine, femoxetine, clomipramine, cianoimipramine, litoxetine, cericlamine, seproxetine, imeldine, ifoxetine, indeloxazine, tiflucarbine, viqualine,milnacipran, bazinaprine, alaproclate, cyanodothepine,trimipramine, quinupramine, dothiepin, Loxapine, nitroxazepine, roxindole, amitriptyline, amitriptyline N-oxide, nortriptyline, pirlindole, indatraline, napamezole, diclofensine, trazodone, sercloremine, nitroquipazine, ademethionine, sibutramine, desmethylsubitramine, didesmethylsubitramine, clovoxamine vilazodone, N-[(1-[(6-Fluoro-2-naphthalenyl)methyl]-4-piperidinyl]amino]carbonyl]-3-pyridine carboxamide (WY 27587), [trans-6-(2-chlorophenyl)-1,2,3,5,6,10b-hexahydropyrrolo-(2,1-a)isoquinoline] (McN 5707), (dl-4-exo-amino-8-chloro-benzo-(b)-bicyclo [3.3.1]nona-2-6 alpha (10 alpha)-diene hydrochloride) (Org 6997), <RTI (dl-[2-[4-(6-fluoro-1H-indol-3-yl)-3,6-dihydro-1(2H)-pyridinyl]ethyl]-3-isopropyl-6-(methylsulphonyl)-3,4-dihydro-1H-2,1,3-benzothiadiazine-2,2-dioxide (LY393558), [4-(5,6-dimethyl-2-benzofuranyl)-piperidine] (CGP 6085), dimethyl-[5-(4-nitro-phenoxy)-6,7,8,9-tetrahydro-5H-benzocyclohepten-7-yl]-amine (RU 25.591), are suitable as SSRIs. The compounds mentioned above may be used in the form of the base or a pharmaceutically acceptable acid addition salt thereof. Each of the serotonin reuptake inhibitors specified above is intended to be an individual embodiment. Accordingly, each of them and the use thereof may be claimed individually.

Other therapeutic compounds which may benefit from combination with compounds of the present invention include compounds, which cause an elevation in the extracellular level of 5-HT in the synaptic cleft. One such compound is tianeptine.

In another embodiment of the present invention, said other medicament is an anti-depressant, such as a tetracyclic antidepressant. Preferred tetracyclic antidepressants include, but are not restricted to, antidepressant drugs in the tetracyclic drug group include: Maprotiline (Ludiomil®), and Mirtazapine, (Remeron® Zispin®).

First-generation antidepressants include the TCAs and the monoamine oxidase inhibitors (MAOs). Both classes of drugs act to reduce the symptoms of depression by increasing the concentrations of noradrenalin and serotonin at the synapse. The neurotransmittance is hereby facilitated by prolonging the effects of noradrenalin and serotonin. Similarly, the second-generation antidepressants such as selective noradrenaline reuptake inhibitors, selective serotonin reuptake inhibitors (SSRI) and selective serotonin and noradrenaline reuptake inhibitors (SSNRI) act to facilitate the neurotransmittance.

Treatment of depression with antidepressants will in a majority of cases result in a significant improvement of symptoms of depression within 3-4 weeks of onset of medication. According to the present invention the period from onset of medication being a combination of at least one compound of the present invention and at least one antidepressant to improvement of symptoms of depression is reduced to about 1 day, such as 2 days, for example 3 days, such as 4 days, for example 5 days, such as 6 days, for example 7 days, such as 8 days, for example 9 days, such as 10 days, for example 11 days, such as 12 days, for example 13 days, such as 14 days, for example 15 days, such as 16 days, for example 17 days, such as 18 days, for example 19 days, such as 20 days, for example 21 days, preferably up to 9 days. In one embodiment of the present invention the treatment of depression comprises the combination of at least one selective serotonine reuptake inhibitor.

In one preferred embodiment, the present invention provides products comprising or consisting of any of the compounds of the present invention and a serotonin reuptake inhibitor as a combination preparation for simultaneous, separate or sequential use in psychiatric drug therapy. Such products may comprise, for example, a kit comprising discrete unit dosage forms containing said compounds and discrete unit dosage forms containing a serotonin reuptake inhibitor, all contained in the same container or pack, e. g. a blister pack.

Without being bound by theory, it is envisaged that the compounds bind to residues in SERT which constitute a functional allosteric site.

Without being bound by theory, it is envisaged that the compounds of the present invention are able to act as antagonists at the SERT, DAT and/or NET, inhibiting binding of cocaine to these transporters without affecting the re-uptake of serotonin, dopamine and/or norepinephrin.

### DETAILED DESCRIPTION OF THE DRAWINGS

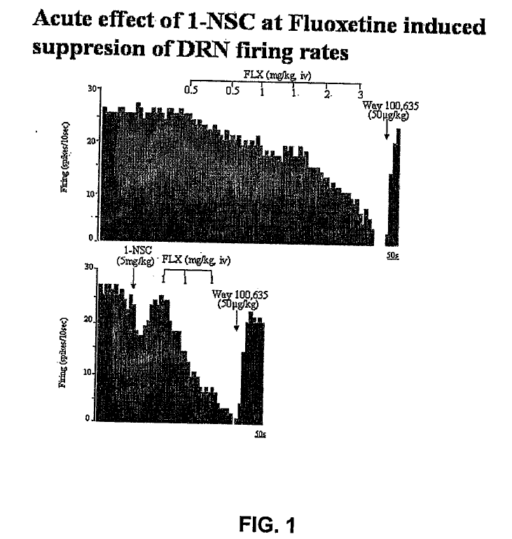

FIG. 1 shows that 1-NSC acutely enhances the fluoxetine-induced suppression of the neuronal firing rate in the dorsal raphe nucleus (DRN). Way 100,635 is a 5HT1A-receptor antagonist that reverses the suppressive effect of fluoxetine on the neuronal firing rate. Additionally, this indicates that the 1-NSC-enhanced suppression is reversible.

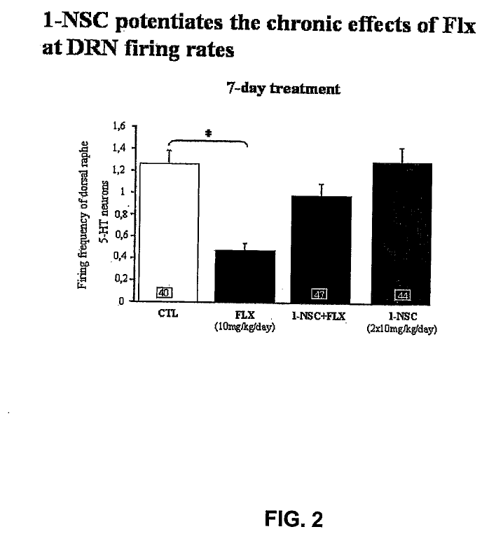

FIG. 2 shows that the recovery of dorsal raphe 5-HT neuronal firing of fluoxetine treated rats, which is believed to be a decisive neurobiolological correlate to clinical recovery is potentiated by 1-NSC. The rats are chronically treated in a 7 day period before firing rates are measured.

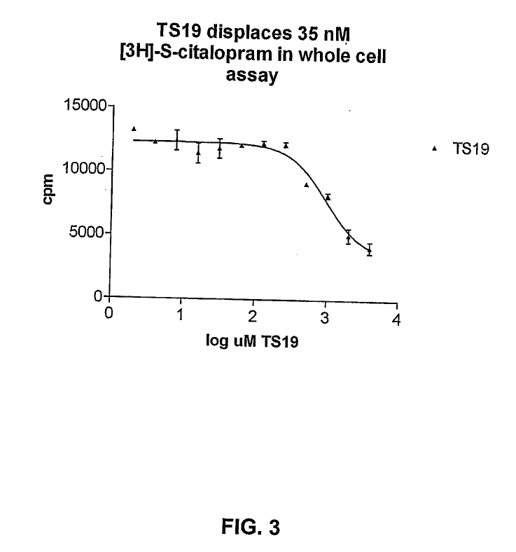

FIG. 3 shows that increasing concentrations of TS19 displaces the radioligand after 60 minutes incubation at room temperature.

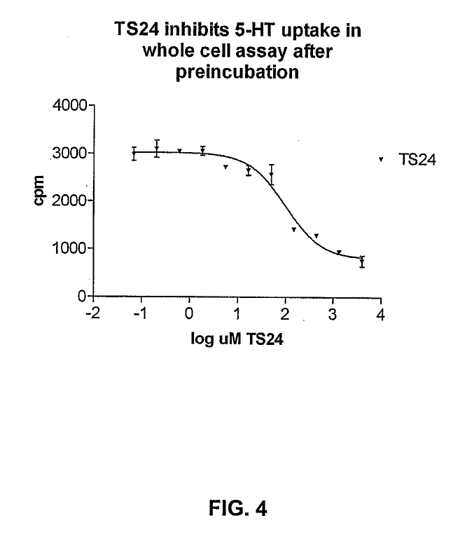

FIG. 4 shows that increasing concentrations of TS24 inhibits [³H]-5-HT uptake in hSERT expressing cells after 60 minutes incubation at room temperature.

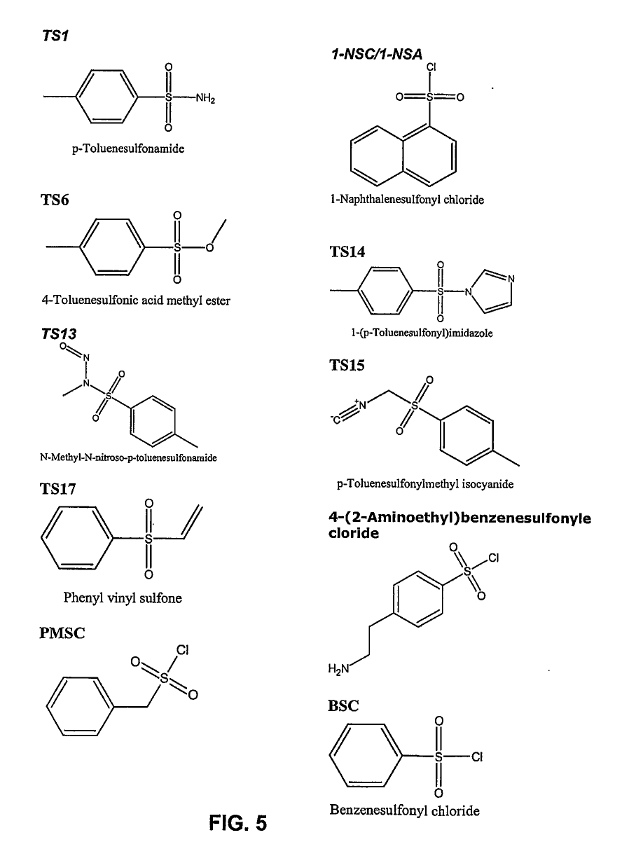

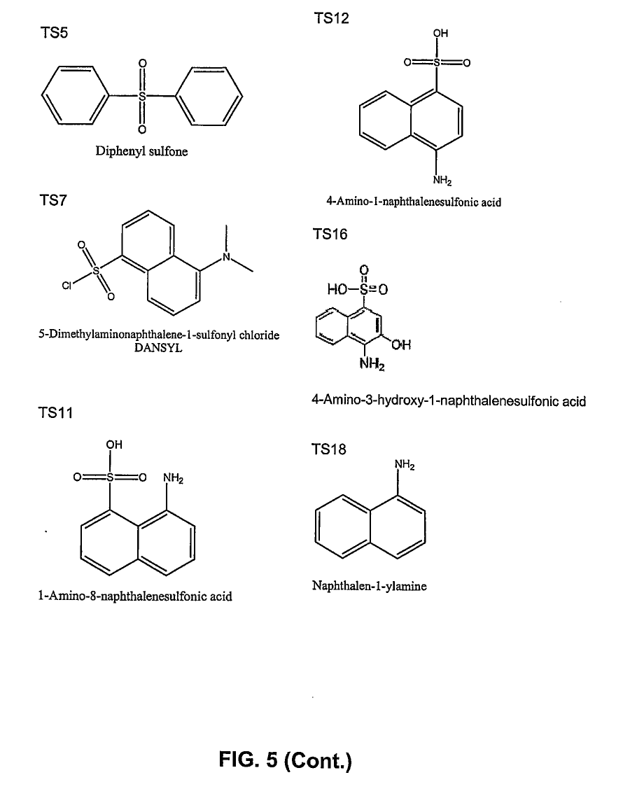

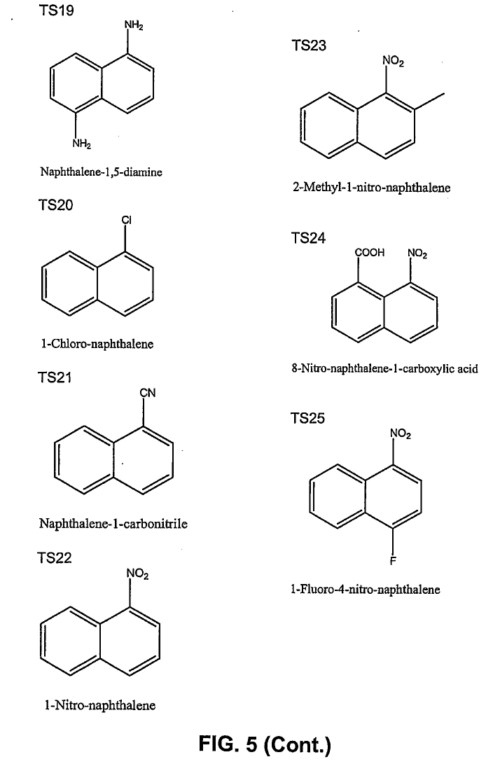

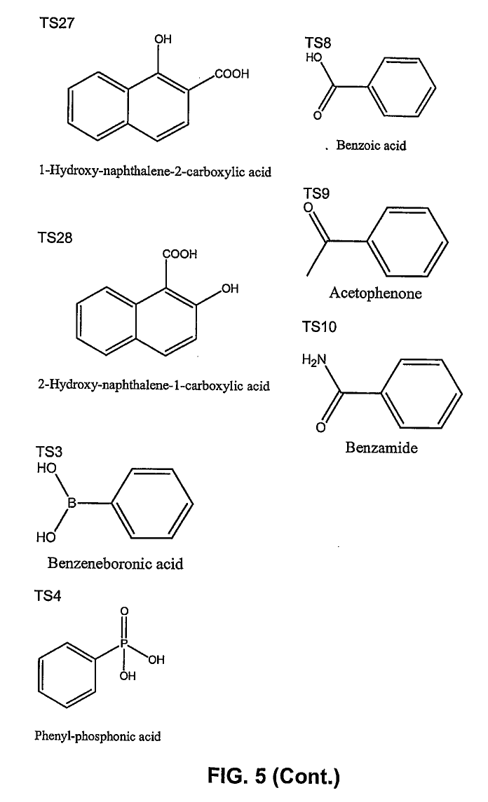

FIG. 5 shows the abbreviations for the compounds of the examples section.

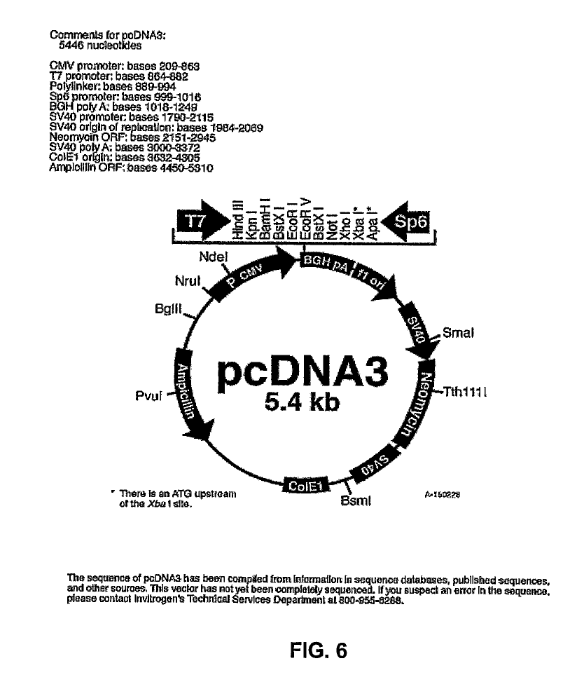

FIG. 6: Schematic representation of the pcDNA3 plasmid vector (Invitrogen).

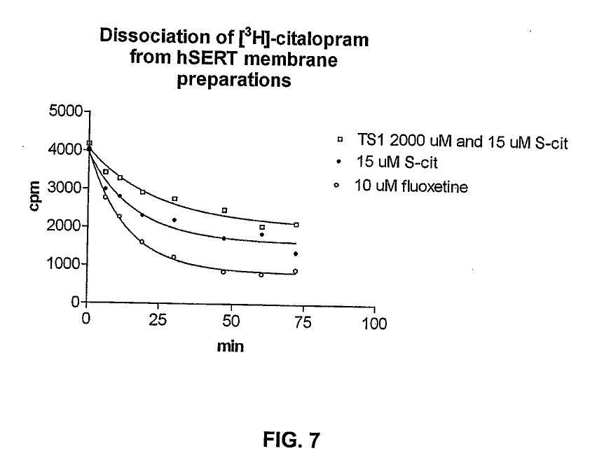

FIG. 7 shows that 2000 μM TS1 increases the allosteric impact on dissociation from membrane preparations by 15 μM citalopram. The allosteric potency of 15 uM citalopram alone can be observed by comparing to the 10 μM fluoxetine curve with no allosteric interaction, because fluoxetine is devoid of allosteric potency. Fluoxetine prevents reassociation of the radioligand.

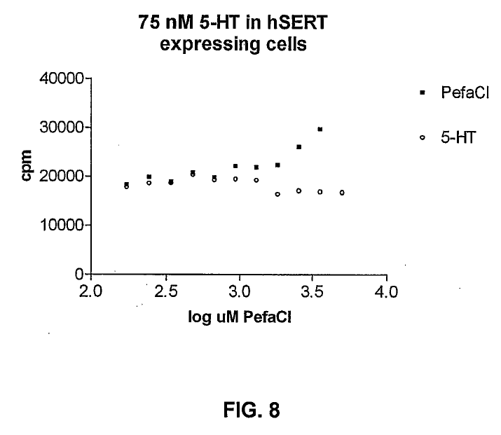

FIG. 8 shows increasing concentrations of PefaCl increases 5-HT uptake ()(10 minutes incubation). For comparison is the baseline 5-HT uptake (∘).

### EXAMPLES

Materials

Dulbecco's modified Eagle's medium, fetal bovine serum, trypsin, and penicillin/streptomycin were purchased from Invitrogen. Cell culture flasks and 96-well plates were from NUNC. White 96-well culture plates and MicroScint-20 scintillation mixture were from Packard. [³H] 5-HT (21.7 Ci/mmol) was from PerkinElmer Life Sciences. [³H]-citalopram (85 Ci/mmol) and citalopram was obtained commercially. Fugene-6 transfection reagent was from Roche Molecular Biochemicals. All chemicals are commercially available. Stock solutions of the allosteric ligands were prepared as 200 mM in DMSO, EtOH or isopropanol.

Transfection Protocols

HEK-293 MSR cells were maintained in Dulbecco's modified Eagle's medium (DMEM) supplemented with 10% fetal calf serum, 0.1 mM nonessential amino acids, 600 μg/mL Geneticin, 100 μg/ml streptomycin, and 100 units/ml penicillin at 37° C. and 5% CO₂ in a humidified atmosphere. The pcDNA3 plasmid vector containing the hSERT gene was used for transfection (see FIG. 6).

5-HT Uptake and Whole-Cell Binding-Assay in 96-Wells Plate

7 μl Fugene-6 was mixed with 112 μl Dulbecco's modified Eagle's medium, and incubated at room temperature for 5 minutes. 4 μg of plasmid were added and the mixture was further incubated at least 15 minutes. Cos-1 cells were trypsinized and suspended in growth media, and the plasmid/Fugene-6 mixture was added followed by dispersion into a 96-well plate. The cells were plated at 35% confluency and grown at 37° C. for 48-64 h.

Membrane Preparations

17.5 μl Fugene-6 (Roche Molecular Biochemicals) were mixed with 280 μl Dulbecco's modified Eagle's medium, and incubated at room temperature for 5 minutes. 10 μg of plasmid were added and the mixture was further incubated at least 15 minutes. HEK-293 MSR cells were trypsinized and suspended in growth media, and the plasmid/Fugene-6 mixture was added. The cells were plated at 35% confluency in a 150 mm dish, and grown at 37° C. for 64 h. Prior to harvesting the dish was rinsed in PBS. Cells were harvested with a cell scraper in buffer 1 (50 mM Tris-base, 150 mM NaCl, 20 mM EDTA, pH 7.4). After centrifugation (3000 g at 4° C. for 10 mins), cells were suspended and homogenized with an IKA Ultra-Turrax from Rose Scientific Ltd (Edmonton, Alberta, Canada) for 20 s in buffer 1. Membranes were pelleted by ultracentrifugation (12 000 g at 4° C. for 15 min) and homogenization was repeated in buffer 1. Finally, after ultracentrifugation (12000 g, 4° C., 15 min) membranes were resuspended in 1 ml buffer 3 (50 mM Tris-base, 120 mM NaCl, 5 mM KCl, pH 7.4) and stored at −80° C.

### Example 1

Measuring the Neuronal Firing Rate In vivo

The experiments were carried out in male Sprague-Dawley rats weighing 250-300 g at the day of the recording. Prior to the acute study, a dose-response curve on the suppressive effect on neuronal firing of fluoxetine was constructed. Subsequently, in the acute study, 5 mg/kg 1-NSC was injected i.v. followed by administration of 1 mg/kg fluoxetine. In the chronic administration experiments, groups of rats were treated with fluoxetine (10 mg/kg/day), 1-NSC (2×10 mg/kg/day) or in combination for 7 days delivered subcutaneously (s.c.) with osmotic minipumps. Control rats received a minipump containing vehicle (NaCl 0.9%). The rats were tested with the minipumps in place.

Extracellular Unitary Recordings of Dorsal Raphe 5-HT Neurons

Extracellular recordings were performed with single-barreled glass micropipettes preloaded with fiberglass filaments in order to facilitate filling. The tip was broken back to 2-4 μm and filled with a 2 M NaCl solution saturated with Pontamine Sky Blue to stain the location. The rats were placed in a stereotaxic frame and a burr hole was drilled on the midline, 1 mm anterior to lambda. Presumed DRN 5-HT neurons were encountered over a distance of 1 mm starting immediately below the ventral border of the Sylvius aqueduct. These neurons were identified using the criteria of Aghajanian (1978): a slow (0.5-2.5 Hz) and regular firing rate and long-duration (0.8-1.2 ms) positive action potentials; they were also verified to be located in DRN. WAY 100635 (N-{2-[4(2-methoxyphenyl)-1-piperazinyl]ethyl}-N-(2-pyridinyl)cyclohexanecarboxamide trihydrochloride; 50-100 μg/kg, i.v.) was used to reverse the suppressant effect of fluoxetine or fluoxetine/1-NSC on the firing activity of DRN 5-HT neurons. To determine a putative interaction between the enantiomers of citalopram on the firing activity of 5-HT neurons, a dose of 250 μg/kg (i.v.) of R-citalopram was injected prior to escitalopram (100 μg/kg, i.v.=ED₁₀₀). For the chronic studies, in order to determine the possible changes of the spontaneous firing activity of dorsal raphe 5-HT neurons, four to five electrode descents were carried out through this nucleus, in rats with minipumps in place.

The allosteric ligand 1-NSC was tested in vivo in Sprague-Dawley rats for the ability to interfere with fluoxetine-induced suppression of the neuronal firing rate (Assay 6). 1-NSC was dissolved in cyclodextrine and injected i.p. When 1-NSC was injected, there was a small decrease in neuronal firing rate, which was restored within minutes (FIG. 1). However, 1-NSC acted by potentiation of the fluoxetine-induced suppression of the neuronal firing rate. The chronic treatment study (FIG. 2) showed that 1-NSC/fluoxetine-treated rats had 80% recovery of the firing rates, compared to the fluoxetine-treated group which had 30% recovery. This raises the possibility that 1-NSC can accelerate the fluoxetine-induced suppression of neuron firing rates and induce a faster recovery of firing rates. A similar potentiation (acute and chronic) was also seen when the rats were treated with escitalopram (data not shown). The doses used in the experiments presented on FIGS. 1 and 2 had no apparent toxic side effects. Additionally, the 5HT1A receptor antagonist Way 100,635 completely restored the neuronal firing rate, indicating that the effect of 1-NSC is reversible.

### Example 2

Binding Assay on Whole Cells Transfected with hSERT

HEK-293 MSR cells were transfected with hSERT as described above, and plated in 96-well plates. The 96-well plate was washed with PBSCM to remove DMEM prior to the assay. 50 μl PBSCM containing 35 nM [³H]-citalopram and increasing concentrations of the allosteric ligands were added to each well. The plate was subsequently incubated for 60 min at room temperature. The assay was terminated by washing once with PBSCM. The amount of accumulated [³H]-citalopram was determined by solubilizing cells in scintillant (MicroScint 20) with direct counting of plates in a Packard TopCounter. Data was analyzed by GraphPad software.

The cells were incubated with increasing concentrations of allosteric ligand and 35 nM [³H]-citalopram. We observed that several of the allosteric ligands displaced [³H]-citalopram with an EC₅₀ in the μM-range (Table 1, FIG. 3). The term “>2000 uM” does not mean that no displacing takes place, but rather that the affinity is in the mM-range.

TABLE 1
whole cell assay with 45 min preincubation

| | | Binding (uM) | 5-HT uptake (uM) |
|---|---|---|---|
| | TS1 | >2000 | >2000 |
| | TS3 | 1424.7 | 735 |
| | TS4 | >2000 | >2000 |
| | TS5 | >2000 | >2000 |
| | TS6 | >2000 | >2000 |
| | TS7 | 1388.0 | >2000 |
| | TS8 | >2000 | >2000 |
| | TS9 | 990.0 | >2000 |
| | TS10 | >2000 | 1149 |
| | TS11 | ND | >2000 |
| | TS12 | 523.0 | ND |
| | TS13 | >2000 | >2000 |
| | TS14 | 1258.8 | 1824 |
| | TS15 | >2000 | >2000 |
| | TS16 | 54.0 | 453 |
| | TS17 | 1049.0 | >2000 |
| | TS18 | 1033.0 | 457.6 |
| | TS19 | 949.9 | 422.3 |
| | TS20 | >2000 | 372.8 |
| | TS21 | >2000 | 60.0 |
| | TS22 | >2000 | 2.9 |
| | TS23 | 1023.0 | 15.9 |
| | TS24 | >2000 | 97.7 |
| | TS25 | >2000 | 25.9 |
| | TS27 | 185.0 | 275.0 |
| | TS28 | 276.0 | >2000 |
| | BSC | ND | ND |
| | PMSC | ND | ND |
| | PefaCI | 1651 | ND |
| Isoprop | 1-NSC | 705 | 283 |
| EtOH | 1-NSC | 533 | 110 |

### Example 3

Binding Assays with Cocaine Analogue [¹²⁵I] RTI-55

A binding assay as described in example 2, wherein the cocaine analogue [¹²⁵I] RTI-55 was used to test for the compounds of the present invention to act as displacers of cocain.

Because RTI-55 (and cocaine) acts as inhibitors of SERT and DAT, we also performed the binding assay in cells transfected with human DAT. As can be seen in Table 2, some of the ligands tested displaced RTI-55 in both transporters. This finding shows that the compounds of the present invention can be used as a cocaine antagonist without uptake inhibitory potency.

TABLE 2
Binding of [125I]-RTI-55 in whole cell assay

| | hDAT (uM) | hSERT (uM) |
|---|---|---|
| 1-NSC react. | ND | 462 |
| EtOH | | |
| 1-NSC react. | | ????? |
| isopropanol | | |
| TS3 | >2000 | >2000 |
| TS9 | >2000 | >2000 |
| TS12 | 212 | 269 |
| TS14 | 1277 | 2314 |
| TS16 | 2.9 | 1.1 |
| TS27 | 138 | 220 |
| TS28 | 288 | 510 |

### Example 4

5-HT Uptake Assay with Preincubation.

The 96-well plate was washed with PBSCM to remove DMEM prior to the assay. 50 μl PBSCM containing increasing concentrations of allosteric ligand were added to each well, and incubated for 60 min at room temperature under moderate agitation. Further 50 μl PBSCM containing 150 nM [³H]-5-HT and increasing concentrations of the allosteric ligands were added to each well, increasing the total buffer volume to 100 μl. The final concentration of [³H]-5-HT was 75 nM and the concentration of the allosteric ligands as unaltered. The plate was subsequently incubated for 10 min at room temperature. The assay was terminated by washing once with PBSCM. The amount of accumulated [³H]-5-HT was determined by solubilizing cells in scintillant (MicroScint 20) with direct counting of plates in a Packard TopCounter. Data was analyzed by GraphPad software.

We tested the allosteric ligands for their ability to inhibit/activate 5-HT-uptake in the whole-cell assay after 60 minutes preincubation. We observed that several of the ligands acted as inhibitors after 60 minutes of preincubation (Table 1, FIG. 4). No ligands were found to increase 5-HT uptake after preincubation. However, we observed that shorter incubation time decreased the inhibitory potency (data not shown). Thus, when adding the allosteric ligand, there is an initial increase in 5-HT uptake followed by inhibition.

The compounds of the examples and their abbreviations are shown in FIG. 5.

### Example 5

Kinetic Assays

Dissociation assay with [³H]-citalopram and membrane preparations SERT-[³H]-citalopram complex was formed by incubating hSERT membrane preparation and radioligand in buffer 3 during a 60 min incubation at 4° C. Radioligand was present at a concentration 10 times the K_{d} value. The time kinetic of dissociation was followed by adding 10 μL complex solution to 250 μL buffer 3 in 96-well plates and incubating subsequently for increasing time intervals at RT. Reactions were terminated by filtration through GF/C glass-fibre filters (Unifilter, Perkin Elmer Life Sciences), preincubated with 40 μl 0.5 % polyethyleneimine, using a Pachard Bell cell harvester, and subsequently washed three times with water. Filters were soaked in 40 μl Microscint 20 scintillation liquid (Pachard Bell). Bound radioactivity was determined by direct counting of plates using a Packard Bell microplate scintillation counter. Dissociation curves were obtained by plotting residual binding vs. time of dissociation.

The allosteric ligands were tested for the ability to affect the dissociation of [³H]-Citalopram in the presence of 15 μM citalopram or 10 μM fluoxetine. Citalopram is an allosteric effector against [³H]-citalopram, and acts by lowering the off-rate. In contrast, fluoxetine is devoid of allosteric potency against. The interaction of the allosteric ligand with the allosteric mechanism used by citalopram was studied by combining allosteric ligand and citalopram in the dissociation buffer. The interaction of the allosteric ligand with the radioligand was studied by combining the allosteric ligand and fluoxetine in the dissociation buffer. Fluoxetine prevents the reassociation of radioligand.

5-HT Uptake Assay

HEK-293 MSR cells were transfected as described in the previous chapter, and plated in 96-well plates. Cells were grown for 48 h to confluency, and prior to uptake assay, plates were washed in PBSCM. All washing steps were carried out in an automatic plate washer. For the determination of potency in 5HT-uptake inhibition, the cells were incubated for 10 min at RT in PBSCM containing 75 nM [³H]-5HT and increasing concentrations of allosteric ligand. The assay was terminated by washing with PBSCM. The amount of accumulated [³H]-5HT was determined by solubilizing cells in scintillant (MicroScint 20) with direct counting of plates in a Packard TopCounter. Data was analyzed by GraphPad software.

Binding of [³H]-Citalopram to Membrane Preparations

A dilution series of the allosteric ligand was prepared in buffer 3 containing 70 nM [³H]-citalopram. Subsequently, 30 μl of the dilution was mixed with 30 μl hSERT membrane preparation homogenate to a final concentration of 35 nM [³H]-citalopram. The mixture was incubated under moderate agitation at RT for 60 min. Reactions were terminated by filtration through GF/C glass-fibre filters (Unifilter, Perkin Elmer Life Sciences), preincubated with 40 μl 0.5 % polyethyleneimine, using a Pachard Bell cell harvester, and subsequently washed three times with water. Filters were soaked in 40 μl Microscint 20 scintillation liquid (Pachard Bell). Bound radioactivity was determined by direct counting of plates using a Packard Bell microplate scintillation counter. Displacing curves were obtained by plotting residual binding vs. log concentration of the displacing ligand.

Results

Dissociation of [³H]-citalopram from the serotonin transporter has been studied using membrane preparations from SERT-expressing cells. The off-rates of the radioligand were measured in the presence/absence of a fixed concentration of allosteric ligand. The interaction with the allosteric mechanism used by citalopram was studied by combining 15 μM citalopram and 200/2000 μM allosteric ligand in the dissociation buffer. Additionally, the allosteric impact on the radioligand in the absence of citalopram, was studied by combining 10 μM fluoxetine and 200/2000 μM allosteric ligand in the dissociation buffer. Fluoxetine is devoid of allosteric potency against [³H]-citalopram, and prevents reassociation of the radioligand. We identified a number of ligands that decreased the off-rate of [³H]-citalopram from the serotonin transporter in the presence/absence of citalopram in the dissociation buffer (Table 3, FIG. 7). Thus, most of these ligands could act as allosteric effectors on their own. This indicate that they use an allosteric mechanism distinct from the one used by citalopram. We identified several allosteric ligands which affected the 5-HT uptake of the serotonin transporter. This was done by adding 50 ul PBSCM containing increasing concentrations of allosteric ligand and 75 nM [3H]-5-HT to each well in a 96-well plate containing SERT-expressing cells. The cells were incubated for 10 minutes before the assay was terminated by washing in PBSCM. In several cases an increase in 5-HT-uptake was observed (Table 3, FIG. 8), whereas other ligands inhibited uptake or was devoid of activity. The ligands had an estimated affinity in the mM-range for affecting 5-HT uptake. The ligands apparently interfered with the 5-HT uptake properties of the transporter.

TABLE 3

| | Stabilises dissociation of | | | Binding of |
|---|---|---|---|---|
| | [³H]-citalopram* | | | [³H]- |
| | 15 uM | Own effect (10 | 5-HT | citalopram |
| | citalopram | uM fluoxetine) | uptake | (Bₘₐₓ) |
| 1-NSC(DMSO) | 2000 uM | 2000 uM | Increase | Displace |
| 1-NSC react. | 300 uM | 300 uM | Inhibition | Displace |
| EtOH | | | | |
| (=1-Naphthalene | | | | |
| sulfonic acid | | | | |
| ethyl ester) | | | | |
| 1-NSC react. | 500 uM | 500 uM | Inhibition | Displace |
| isopropanol | | | | |
| (=1-Naphthalene | | | | |
| sulfonic acid | | | | |
| isopropenyl | | | | |
| ester) | | | | |
| PefaCI | 2000 uM | 2000 uM | Increase | No effect |
| | | | (FIG. 8) | |
| PMSC | No effect | ND | Increase | No effect |
| BSC | 3000 uM | ND | Increase | No effect |
| TS1 | 200 uM | 2000 uM | Increase | No effect |
| | (FIG. 7) | | | |
| TS6 | 2000 uM | No effect | Increase | No effect |
| TS13 | 200 uM | 200 uM | Increase | displace |
| TS14 | 2000 uM | 2000 uM | No effect | No effect |
| TS15 | 2000 uM | 2000 uM | No effect | No effect |
| TS17 | 2000 uM | 2000 uM | ND | No effect |
| TS20 | 200 uM | 200 uM | Inhibition | No effect |
| TS22 | 200 uM | 200 uM | Inhibition | No effect |

*The presented value is the concentrations which affect dissociation of [³H]-citalopram

1. A method of treating a CNS disorder in an individual, wherein said method comprises administering a compound, or a pharmaceutically acceptable salt or a pharmaceutically acceptable acid addition salt thereof, in combination with another medicament for a CNS disorder, to said individual in need of such treatment in a therapeutically effective amount: and
wherein said compound has the general formula A 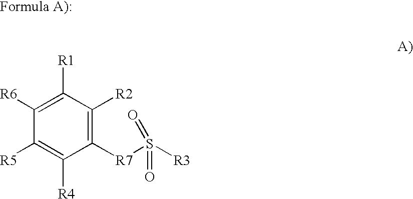
wherein R1, R2, R4, R5 are independently selected from any of the following chemical groups:
H, F, Cl, Br, I, hydroxy, alkoxy, alkenoxy, aryloxy, carbamoyl, carboxyl, thiol groups, alkyl sulfide, aryl sulfide, sulfone, sulfonyl, sulfoxide, amine, alkylamine, dialkylamine, arylamine, alkylarylamine, diarylamine, N-oxides (for example nitro-), mercapto, cyano, a hydrocarbon substituent selected from the group consisting of:
alkyl, substituted alkyl,
cyclic alkyl, substituted cyclic alkyl,
aryl, substituted aryl,
alkenyl, substituted alkenyl,
alkynyl, substituted alkynyl,
aralkyl, substituted aralkyl,
heterocyclyl, substituted heterocyclyl,
heterocyclylalkyl, substituted heterocyclylalkyl,
alkylaminoalkyl, substituted alkylaminoalkyl,
dialkylaminoalkyl, substituted dialkylaminoalkyl,
heterocyclyloxyalkyl, substituted heterocyclyloxyalkyl,
arylaminoalkyl, substituted arylaminoalkyl,
heterocyclylaminoalkyl, substituted heterocyclylaminoalkyl,
alkylaminoalkoxy, substituted alkylaminoalkoxy,
dialkylaminoalkoxy, substituted dialkylaminoalkoxy,
heterocyclyloxy, and substituted heterocyclyloxy;
and wherein R3 is selected from the group consisting of:
H, Cl, Br, I, hydroxy, alkoxy, alkyl, substituted alkyl, cyclic alkyl, substituted cyclic alkyl, alkenyl, substituted alkenyl, alkynyl, substituted alkynyl, aralkyl, and substituted aralkyl, and wherein R6 is selected from the group consisting of:
H, F, Cl, Br, I, hydroxy, alkoxy, aryloxy, carbamoyl, carboxyl, thiol, alkyl sulfide, aryl sulfide, sulfone, sulfonyl, sulfoxide, amine, alkylamine, dialkylamine, arylamine, alkylarylamine, diarylamine, mercapto, cyano,
alkyl, substituted alkyl,
cyclic alkyl, substituted cyclic alkyl,
aryl, substituted aryl,
alkenyl, substituted alkenyl,
alkynyl, substituted alkynyl,
aralkyl, substituted aralkyl,
heterocyclyl,
heterocyclylalkyl, substituted heterocyclylalkyl,
alkylaminoalkyl, substituted alkylaminoalkyl,
dialkylaminoalkyl, substituted dialkylaminoalkyl,
heterocyclyloxyalkyl, substituted heterocyclyloxyalkyl,
arylaminoalkyl, substituted arylaminoalkyl,
heterocyclylaminoalkyl, substituted heterocyclylaminoalkyl,
alkylaminoalkoxy, substituted alkylaminoalkoxy,
dialkylaminoalkoxy, substituted dialkylaminoalkoxy,
heterocyclyloxy, and substituted heterocyclyloxy,
and wherein —R7- is a single bond;
and wherein either one or none of the following pairs of R groups form part of an additional benzene ring:
(R1 and R2)
(R1 and R6)
(R5 and R6)
(R4 and R5);
wherein said additional benzene ring is optionally a substituted benzene ring;
with the provisos for formula A that:
if R3 comprises a five-sided organic ring, then R3 does not comprise a six-sided organic ring; and
if R6 comprises a five-sided organic ring, then R6 does not comprise a six-sided organic ring; and
if R6 comprises a linear carbon chain or substituted linear carbon chain, then said carbon chain is C₁₋₇, and
wherein the compound is not 5-dimethylaminonaphtalene-1-sulfonyl chloride.

2. The method according to claim 1, wherein said CNS disorder is selected from the group consisting of: anxiety, social anxiety disorder, generalized anxiety disorder, post-traumatic stress disorder, panic disorder, a sleep disorder, an eating disorder, or obsessive compulsive disorder (OCD).

3. The method according to claim 1, wherein said CNS disorder is a psychiatric disease, such as selected from the group consisting of depression, anxiety, mania, ecstasy syndrome, obsessive compulsory disorder (OCD) and/or eating disorders.

4. The method according to claim 1, wherein said CNS disorder is depression.

5. (canceled)

6. The method according to claim 1, wherein said compound has a molecular weight below 10,000 Da.

7. The method according to claim 1, wherein R1, R2, R4, and R5 independently are selected from the group consisting of:
H, F, Cl, Br, I, hydroxy, alkoxy, aryloxy, carbamoyl, carboxyl, thiol, alkyl sulfide, aryl sulfide, sulfone, sulfonyl, sulfoxide, amine, alkylamine, dialkylamine, arylamine, alkylarylamine, diarylamine, N-oxide, mercapto, cyano,
alkyl, substituted alkyl,
cyclic alkyl, substituted cyclic alkyl,
aryl, substituted aryl,
alkenyl, substituted alkenyl,
alkynyl, substituted alkynyl,
aralkyl, substituted aralkyl,
heterocyclyl, substituted heterocyclyl,
heterocyclylalkyl, substituted heterocyclylalkyl,
alkylaminoalkyl, substituted alkylaminoalkyl,
dialkylaminoalkyl, substituted dialkylaminoalkyl,
heterocyclyloxyalkyl, substituted heterocyclyloxyalkyl,
arylaminoalkyl, substituted arylaminoalkyl,
heterocyclylaminoalkyl, substituted heterocyclylaminoalkyl,
alkylaminoalkoxy, substituted alkylaminoalkoxy,
dialkylaminoalkoxy, substituted dialkylaminoalkoxy,
heterocyclyloxy, and substituted heterocyclyloxy,

8. The method according to claim 1, wherein if R1, R2, R3, R4, R5, and R6 independently comprises a linear carbon skeleton, then said linear carbon skeleton is C₁₋₅.

9. The method use according to claim 1, wherein R1, R2, R4, R5, and R6 independently is selected from the group consisting of: H, F, Cl, Br, I, hydroxyl and alkoxy.

10. The method according to claim 1, wherein R1, R2, R4, R5, and R6 independently is selected from the group consisting of: aryloxy, carbamoyl, carboxyl, thiol, alkyl sulphide, aryl sulphide, sulfone, sulfonyl, sulfoxide or amine.

11. The method according to claim 1, wherein R1, R2, R4, and R5 independently is selected from the group consisting of: alkylamine, dialkylamine, arylamine, alkylarylamine, diarylamine, or an N-oxide.

12. The method according to claim 1, wherein R1, R2, R4, R5, and R6 independently is selected from the group consisting of: alkyl, substituted alkyl, cyclic alkyl, and substituted cyclic alkyl.

13. The method according to claim 1, wherein R1, R2, R4, R5, and R6 independently is selected from the group consisting of: aryl or substituted aryl.

14. The method according to claim 1, wherein R1, R2, R4, R5, and R6 independently is selected from the group consisting of: alkenyl, substituted alkenyl, alkynyl, substituted alkynyl, aralkyl, or substituted aralkyl.

15. The method according to claim 1, wherein R1, R2, R4, and R5 independently is selected from the group consisting of: heterocyclyl, substituted heterocyclyl, heterocyclylalkyl, substituted heterocyclylalkyl, alkylaminoalkyl, substituted alkylaminoalkyl, dialkylaminoalkyl, substituted dialkylaminoalkyl, heterocyclyloxyalkyl, or substituted heterocyclyloxyalkyl.

16. The method according to claim 1, wherein R1, R2, R4, R5, and R6 independently is selected from the group consisting of: arylaminoalkyl, substituted arylaminoalkyl, heterocyclylaminoalkyl, or substituted heterocyclylaminoalkyl.

17. The method according to claim 1, wherein R1, R2, R4, R5, and R6 independently is selected from the group consisting of: alkylaminoalkoxy, substituted alkylaminoalkoxy, dialkylaminoalkoxy, substituted dialkylaminoalkoxy, heterocyclyloxy, or substituted heterocyclyloxy.

18. The method according to claim 1, wherein R1, R2, R3, R4, R5, and R6 independently is H.

19. The method according to claim 7, wherein the optionally substituted alkyl is a straight chain alkyl selected from the group consisting of: methyl, ethyl, propyl, butyl, pentyl, hexyl, heptyl, octyl, nonyl, decyl, undecyl, and dodecyl.

20. The method according to claim 7, wherein the optionally substituted alkyl is a branched chain alkyl selected from the group consisting of:
—CH(CH₃)₂,
—CH(CH₃)(CH₂CH₃),
—CH(CH₂CH₃)₂,
—C(CH₃)₃,
—C(CH₂CH₃)₃,
—CH₂CH(CH₃)₂,
—CH₂CH(CH₃)(CH₂CH₃),
—CH₂CH(CH₂CH₃)₂,
—CH₂C(CH₃)₃,
—CH₂C(CH₂CH₃)₃,
—CH(CH₃)CH(CH₃)(CH₂CH₃),
—CH₂CH₂CH(CH₃)₂,
—CH₂CH₂CH(CH₃)(CH₂CH₃),
—CH₂CH₂CH(CH₂CH₃)₂,
—CH₂CH₂C(CH₃)₃,
—CH₂CH₂C(CH₂CH₃)₃,
—CH(CH₃)CH₂CH(CH₃)₂,
—CH(CH₃)CH(CH₃)CH(CH₃)CH(CH₃)₂, and
—CH(CH₂CH₃)CH(CH₃)CH(CH₃)(CH₂CH₃).

21. The method according to claim 7, wherein the optionally substituted cycloalkyl is selected from the groups consisting of: cyclopropyl, cyclobutyl, cyclopentyl, cyclohexyl, cycloheptyl, and cyclooctyl and rings substituted with straight and branched chain alkyl groups, such as defined in any of the preceding claims.

22. The method according to claim 7, wherein the optionally substituted cycloalkyl is a C₅-C₁₆ cycloalkyl group.

23. The method according to claim 7, wherein the substituted hydrocarbon substituent is substituted with one or more, such as two, three, four, five, six, seven, eight, nine and ten, of a halogen atom such as F, Cl, Br, and I; and oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as in trialkylsilyl groups, dialkylarylsilyl groups, alkyldiarylsilyl groups, and triarylsilyl groups.

24. The method according to claim 7, wherein the substituted hydrocarbon substituent is substituted with one or more, such as two, three, four, five, six, seven, eight, nine and ten, of a heteroatom, such as oxygen in carbonyl, carboxyl, and ester groups; nitrogen in groups such as imines, oximes, hydrazones, and nitriles.

25. The method according to claim 7, wherein the substituted hydrocarbon substituent contains a hydroxyl, alkoxy, aryloxy group, or heterocyclyloxy group.

26. The method according to claim 7, wherein the substituted hydrocarbon substituent contains an amine, alkylamine, dialkylamine, arylamine, (alkyl)(aryl)amine, diarylamine, heterocyclylamine, (alkyl)(heterocyclyl)amine, (aryl)(heterocyclyl)amine, or diheterocyclylamine group.

27. The method according to claim 7, wherein the substituted hydrocarbon substituent is an aliphatic functional group substituted with an aryl group such as an (C6-C12) aryl group.

28. The method according to claim 7, wherein the substituted hydrocarbon substituent is a substituted aryl group, such as an aralkyl group, which can be substituted or non-substituted.

29. The method according to claim 7, wherein the (optionally substituted) aralkyl group is selected from benzyl, diphenylmethyl, 1-phenylethyl(-CH(C₆H₅)(CH₃)), 2-phenylethyl group, and 2-naphthylethyl group.

30. The method according to claim 7, wherein the (optionally substituted) alkyl residue comprises or consists of a C₅-C₂₀ alkyl group.

31. The method according to claim 7, wherein the alkyl is substituted with an aryl or heteroaryl residue.

32. The method according to claim 7, wherein the alkyl substituted with an aryl or heteroaryl residue is selected from the group consisting of linear (C1-C10) alkyls, branched (C4-C10) alkyls, cyclic (C5-C10) alkyls, such as a methyl group, ethyl group, propyl group, such as a n-propyl group and an isopropyl group, butyl group, such as n-butyl group, isobutyl group, t-butyl group, n-amyl group, pentyl group, such as neopentyl group, cyclopentyl group, hexyl group, such as n-hexyl group, cyclohexyl group, heptyl group, octyl group, such as n-octyl group, nonyl group, such as n-nonyl group, decyl group, such as n-decyl group, undecyl group, dodecyl group, and mentyl group.

33. The method according to claim 7, wherein the alkyl is an optionally substituted C₅-C20 alkyl group which, when substituted, is substituted with one or more of a halogen atom, such as fluorine atom, a chlorine atom, a bromine atom, and an iodine atom, an alkoxy group, such as a (C1-C4) alkoxy group such as methoxy group, ethoxy group, n-propoxy group, t-butoxy group, an aryloxy group, such as phenoxy, an alkylthio group, such as n-propylthio and t-butylthio, and an arylthio group, such as phenylthio.

34. The method according to claim 7, wherein the optionally substituted alkenyl is a straight or branched chain or a cyclic group, such as vinyl,
—CH═C(H)(CH₃), —CH═C(CH₃)₂, —C(CH₃)═C(H)₂, —C(CH₃)═C(H)(CH₃), —C(CH₂CH₃)═CH₂, cyclohexenyl, cyclopentenyl, cyclohexadienyl, butadienyl, pentadienyl, and hexadienyl.

35. The method according to claim 7, wherein the-substituted alkenyl group includes alkenyl groups in which a non-carbon or non-hydrogen atom is bonded to a carbon double bonded to another carbon and those in which one of the non-carbon or non-hydrogen atoms is bonded to a carbon not involved in a double bond to another carbon.

36. The method according to claim 7, wherein the alkynyl is a straight or branched chain group such as —CC(H), —CC(CH₃), —CC(CH₂CH₃), —C(H₂)CC(H), —C(H)₂CC(CH₃), and —C(H)₂CC(CH₂CH₃).

37. The method according to claim 7, wherein the substituted alkynyl includes alkynyl groups in which a non-carbon or non-hydrogen atom is bonded to a carbon triple bonded to another carbon and those in which a non-carbon or non-hydrogen atom is bonded to a carbon not involved in a triple bond to another carbon.

38. (canceled)

39. (canceled)

40. (canceled)

41. (canceled)

42. (canceled)

43. The method according to claim 1, wherein at least 4 of R1-R6 have a molecular weight of less than 30, for example less than 20, such as less than 15.

44. The method according to claim 1, wherein at least 4 of R1-R6 are H.

45. The method according to method claim 1, wherein at least 4 of R1-R6 are selected from the group consisting of: H, OH, NH, and a halogen.

46. The method according to claim 1, wherein said compounds are selected from the group consisting of:
1-amino-8-naphtalenesulfonic acid, 4-amino-1-naphtalenesulfonic acid, 4-amino-3-hydroxy-1-naphthalenesulfonic acid, 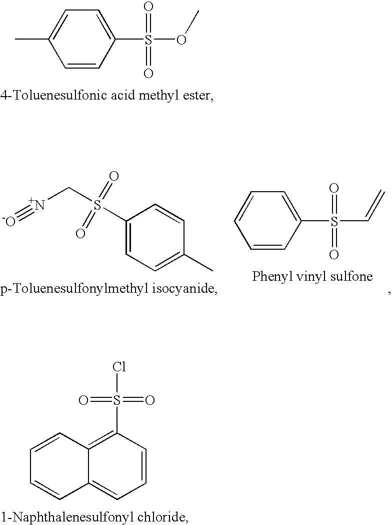
1-naphthalenesulfonic acid,
Naphthalene-1-sulfonic acid isopropyl ester,
Naphthalene-1-sulfonic acid isopropenyl ester,
Naphthalene-1-sulfonic acid ethyl ester,

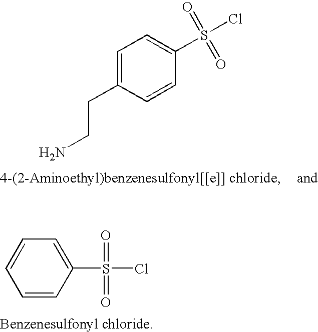

47. The method according to claim 46, wherein said compounds are selected from the group consisting of: 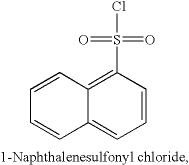
1-naphthalenesulfonic acid,
Naphthalene-1-sulfonic acid isopropyl ester, and
Naphthalene-1-sulfonic acid ethyl ester.

48. The method according to claim 1, wherein said compound is in a formulation suitable for oral or subcutaneous administration.

49. The method according to claim 1, wherein said compound is administered in a dosage of 1-150 mg/day, such as 100 mg/day.

50. The method according to claim 1, wherein said another medicament is an anti-depressant.

51. The method according to claim 50, wherein said anti-depressant is a selective serotonin reuptake inhibitor.

52. The method according to claim 50, wherein the combination is administered in order to speed up the onset of the anti-depressant effect in a synergistic manner.

53. The method according to claim 50, wherein the period from onset of medication, being a combination of at least one compound of formula A) and at least one antidepressant, to improvement of symptoms of depression, is reduced to about up to 9 days, such as 10 days, for example 11 days, such as 12 days, for example 13 days, such as 14 days, for example 15 days, such as 16 days, for example 17 days, such as 18 days, for example 19 days, such as 20 days, or for example 21 days.

54. (canceled)

55. (canceled)

56. (canceled)

57. A method of treating pain in an individual, said method comprises administering a compound, or a pharmaceutically acceptable salt or a pharmaceutically acceptable acid addition salt thereof, to said individual in need of such treatment in a therapeutically effective amount; and
wherein said compound has the general formula A 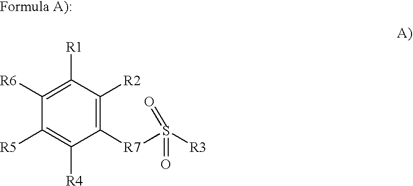
wherein R1, R2, R4, R5 are independently selected from any of the following chemical groups:
H, F, Cl, Br, I, hydroxy, alkoxy, alkenoxy, aryloxy, carbamoyl, carboxyl, thiol groups, alkyl sulfide, aryl sulfide, sulfone, sulfonyl, sulfoxide, amine, alkylamine, dialkylamine, arylamine, alkylarylamine, diarylamine, N-oxides (for example nitro-), mercapto, cyano, a hydrocarbon substituent selected from the group consisting of:
alkyl, substituted alkyl,
cyclic alkyl, substituted cyclic alkyl,
aryl, substituted aryl,
alkenyl, substituted alkenyl,
alkynyl, substituted alkynyl,
aralkyl, substituted aralkyl,
heterocyclyl, substituted heterocyclyl,
heterocyclylalkyl, substituted heterocyclylalkyl,
alkylaminoalkyl, substituted alkylaminoalkyl,
dialkylaminoalkyl, substituted dialkylaminoalkyl,
heterocyclyloxyalkyl, substituted heterocyclyloxyalkyl,
arylaminoalkyl, substituted arylaminoalkyl,
heterocyclylaminoalkyl, substituted heterocyclylaminoalkyl,
alkylaminoalkoxy, substituted alkylaminoalkoxy,
dialkylaminoalkoxy, substituted dialkylaminoalkoxy,
heterocyclyloxy, and substituted heterocyclyloxy;
and wherein R3 is selected from the group consisting of:
H, Cl, Br, I, hydroxy, alkoxy, alkyl, substituted alkyl, cyclic alkyl, substituted cyclic alkyl, alkenyl, substituted alkenyl, alkynyl, substituted alkynyl, aralkyl, and substituted aralkyl;
and wherein R6 is selected from the group consisting of:
H, F, Cl, Br, I, hydroxy, alkoxy, aryloxy, carbamoyl, carboxyl, thiol, alkyl sulfide, aryl sulfide, sulfone, sulfonyl, sulfoxide, amine, alkylamine, dialkylamine, arylamine, alkylarylamine, diarylamine, mercapto, cyano,
alkyl, substituted alkyl,
cyclic alkyl, substituted cyclic alkyl,
aryl, substituted aryl,
alkenyl, substituted alkenyl,
alkynyl, substituted alkynyl,
aralkyl, substituted aralkyl,
heterocyclyl,
heterocyclylalkyl, substituted heterocyclylalkyl,
alkylaminoalkyl, substituted alkylaminoalkyl,
dialkylaminoalkyl, substituted dialkylaminoalkyl,
heterocyclyloxyalkyl, substituted heterocyclyloxyalkyl,
arylaminoalkyl, substituted arylaminoalkyl,
heterocyclylaminoalkyl, substituted heterocyclylaminoalkyl,
alkylaminoalkoxy, substituted alkylaminoalkoxy,
dialkylaminoalkoxy, substituted dialkylaminoalkoxy,
heterocyclyloxy, and substituted heterocyclyloxy;
and wherein —R7- is a single bond;
and wherein either one or none of the following pairs of R groups form part of an additional benzene ring:
(R1 and R2)
(R1 and R6)
(R5 and R6)
(R4 and R5);
wherein said additional benzene ring is optionally a substituted benzene ring;
with the provisos for formula A that:
if R3 comprises a five-sided organic ring, then R3 does not comprise a six-sided organic ring; and
if R6 comprises a five-sided organic ring, then R6 does not comprise a six-sided organic ring; and
if R6 comprises a linear carbon chain or substituted linear carbon chain, then said carbon chain is C₁₋₇; and
wherein the compound is not 5-dimethylaminonaphtalene-1-sulfonyl chloride.

58. The method according to claim 57, wherein said compounds are selected from the group consisting of:
1-amino-8-naphtalenesulfonic acid, 4-amino-1-naphtalenesulfonic acid, 4-amino-3-hydroxy-1-naphthalenesulfonic acid, 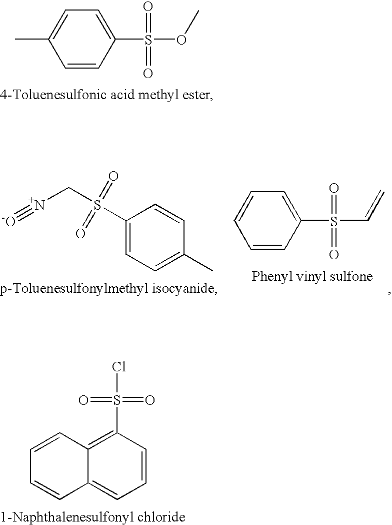
1-naphthalenesulfonic acid,
Naphthalene-1-sulfonic acid isopropyl ester,
Naphthalene-1-sulfonic acid isopropenyl ester,
Naphthalene-1-sulfonic acid ethyl ester,

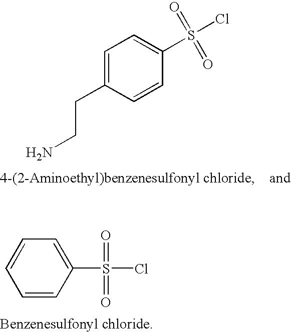

59. The method according to claim 57, wherein said compounds are selected from the group consisting of:, 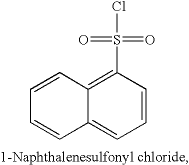
1-naphthalenesulfonic acid,
Naphthalene-1-sulfonic acid isopropyl ester, and
Naphthalene-1-sulfonic acid ethyl ester.
